(19) United States
(12) Patent Application Publication
(10) Pub. No.: US 2009/0162839 A1
(43) Pub. Date: Jun. 25, 2009

(54) DIAGNOSIS AND PROGNOSIS OF CANCER BASED ON TELOMERE LENGTH AS MEASURED ON CYTOLOGICAL SPECIMENS

(75) Inventors: Ruth L. Katz, Houston, TX (US); Jun Gu, Houston, TX (US); Abha Khanna, Houston, TX (US); Ricardo Fernandez, Houston, TX (US)

Correspondence Address: FULBRIGHT & JAWORSKI, LLP, 1301 MCKINNEY, SUITE 5100, HOUSTON, TX, 77010-3095, US

(73) Assignee: Board of Regents, The University of Texas System, Austin, TX (US)

(21) Appl. No.: 11/574,449

(22) PCT Filed: Aug. 31, 2005
(86) PCT No.: PCT/US05/31147

§ 371 Date: Feb. 12, 2009

Related U.S. Application Data
(60) Provisional application No. 60/605,972, filed on Aug. 31, 2004.

(51) Int. Cl. C12Q 1/68

(52) U.S. Cl. 435/6

(57) ABSTRACT

The present invention concerns a quantitative in situ assessment of mean telomere length, particularly in relation to nuclear area, for the diagnosis and/or prognosis of cancer. In particular aspects, the methods and compositions regard diagnosis and/or prognosis of bladder cancer, urothelial cancer, lung cancer, and lymphoma.

This application claims priority to U.S. Provisional Patent Application Ser. No. 60/605,972, filed Aug. 31, 2004, which is incorporated by reference herein in its entirety.

### FIELD OF THE INVENTION

In particular, the present invention relates at least to cell biology, molecular biology, and cancer prognosis and diagnosis. Specifically, the present invention regards telomere length as it relates to cancer prognosis and diagnosis.

### BACKGROUND OF THE INVENTION

Telomeres are specialized protein-bound DNA structures at the ends of eukaryotic chromosomes that appear to function in chromosome stabilization, positioning, and replication (Blackburn and Szostak, 1984; Zakian, 1989; Blackburn, 1991). In all vertebrates, telomeres consist of hundreds to thousands of tandem repeats of a 5′-TTAGGG-3′ sequence and associated proteins (Blackburn, 1991; Moyzis et al., 1988). Southern blot analysis of chromosome terminal restriction fragments (TRF) provides the composite lengths of all telomeres in a cell population (Harley et al., 1990; Allsopp et al., 1992; Vaziri et al., 1993). In all normal somatic cells examined to date, TRF analysis has shown that the chromosomes lose about 50-200 nucleotides of telomeric sequence per cell division, consistent with the inability of DNA polymerase to replicate linear DNA to the ends (Harley et al., 1990; Allsopp et al., 1992; Vaziri et al., 1993; Watson, 1972).

This shortening of telomeres has been proposed to be the mitotic clock by which cells count their divisions (Harley, 1991), and a sufficiently short telomere(s) may be the signal for replicative senescence in normal cells (Allsopp et al., 1992; Vaziri et al., 1993; Hastie et al., 1990; Lindsey et al., 1991; Wright and Shay, 1992). In contrast, the vast majority of immortal cells examined to date shows no net loss of telomere length or sequence with cell divisions, suggesting that maintenance of telomeres is required for cells to escape from replicative senescence and to proliferate indefinitely (Counter et al., 1992; Counter et al., 1994).

In general, telomerase activity is absent in most somatic human cells, however normal and reactive lymphocytes, germ line and tumor cells possess telomerase.

In particular, telomere dysfunction, characterized primarily by shortened telomeres, occurs both in bladder cancer precursor lesions, such as carcinoma in situ (CIS), as well as in papillary and invasive urothelial cancer. Chromosomal instability is a hallmark of urothelial cancer and may occur via shortened telomeres, which permit chromosome end-to-end fusions, and generation of mutlicentric chromosomes that missegregate in mitosis leading to aneusony and structural abnormalities.

U.S. Pat. Nos. 5,693,474 and 5,639,613 describe predicting tumor progression and prognosticating cancer by analyzing a sample suspected of having cancer cells for telomerase activity, wherein a high telomerase activity indicates an unfavorable prognosis. It is noted therein that it is difficult to diagnose lung cancer by cytology alone.

Kageyama et al. (1997) examined telomere lengths by Southern blot and telomerase activity by TRAP assay in vitro in bladder and prostate cancer cell lines. In bladder cancer cell lines, the telomere length decreased with increasing cell passage.

Golubovskaya et al. (1999) assayed telomere length and telomerase activity in rat epithelial stem-like cells in association with chromosome instability. Specifically, FISH was performed with a telomere-specific probe, and it was determined that telomere erosion and telomerase expression has an effect on chromosomal instability.

Dalquen et al. (2002) compared the diagnostic value of DNA image cytometry and fluorescence in situ hybridization (FISH) for detecting urothelial tumors in voided urine in a non-invasive manner. They compared cytospin preparations of benign prostatic hyperplastic patients having noninvasive or invasive tumors with the AUTOCYTE™ cell analytical system on Feulgen-stained samples, with the analysis of certain chromosomes using UroVysion™ FISH probes. UroVysion detects aneuploidy of cells, usually obtained by non-invasive means, and particularly in individuals already diagnosed with bladder cancer. Although both methods were considered successful as supplementary methods to cystoscopic and histological methods, UroVysion FISH was more sensitive for the detection of noninvasive tumors than DNA image cytometry.

Halling et al. (2002) compared sensitivity for detection of urothelial carcinoma utilizing UroVysion (Vysis, Inc.; Downers Grove, Ill.); BTA stat (B.D.S., Inc.; Redmond, Wash.) (a tumor marker immunoassay for bladder tumor-associated antigen, which has been identified as complement factor H related protein (CFHrp)); hemoglobin dipstick; and a telomerase measuring assay using a polymerase chain reaction-based telomere repeat amplification protocol (TRAP) assay. Each of UroVysion, BTA stat, and hemoglobin dipstick were statistically more sensitive than the telomerase activity.

Van Heek et al. (2002) identify telomere shortening in pancreatic intraepithelial neoplasia using telomere-specific FISH and immunostaining.

Sarosdy et al. (2002) evaluated the UroVysion fluorescence in situ hybridization assay (Vysis, Inc.; Downers Grove, Ill.) in comparison to BTA Stat test and cytology and determined it was more sensitive to cytology and about equivalent to the BTA Stat test for detection of recurrent transitional cell carcinoma.

Plentz et al. (2004) determine telomere length of hepatocytes from liver tumors by a combination of histological and cytological methods. Telomere length was shorter in hepatocellular carcinoma samples compared to normal controls, and was significantly shorter in aneuploid tumors compared to diploid tumors. Specifically, quantitative FISH (q-FISH) is employed wherein a telomere-specific Cy-3 probe stained fine-needle hepatocyte samples and quantitated the staining with a telomere analysis software program.

Varella-Garcia et al. (2004) utilized UroVysion FISH analysis of urine specimens being monitored for bladder cancer recurrence, which was compared to urine cytology/flexible cystoscopy analyses. Although the specificity was identical for both methods, the sensitivity was greater for the FISH analysis. The authors note therein at least one tumor sample that was not identified as cancerous by either method.

U.S. Pat. No. 5,707,795 is directed to diagnosing the stage of disease progression based on measuring telomere lengths from cells of an individual having a disease associated with cell proliferation and comparing them to a control. In specific embodiments, the telomere lengths are measured by Southerns, by primer extension, or by measuring signal intensity of a label on a probe specific for telomeric DNA, such as by in situ hybridization and microfluorometry.

U.S. Pat. No. 5,693,474 regards methods of prognosticating cancer by analyzing a sample for telomerase activity, particularly by primer extension methods.

WO 97/35871 relates to detecting bladder cancer by telomerase activity, wherein an increase in telomerase activity confers a positive correlation on the presence of bladder cancer cells in a sample. The detection may comprise primer extension, in certain embodiments. However, in alternative embodiments, the lengths of telomeres may be measured and compared to the lengths of telomeres in cells of the same histologic type contained in a urine sample from a subject matched by age, tumor grade, level of invasion, or any other prognostic indicator.

U.S. Pat. Nos. 6,174,681 and 6,376,188, and U.S. Patent Application Publication No. 2002/0160409 are all directed to the UroVysion FISH method (Vysis Inc.; Downers Grove, Ill.) and compositions, wherein cancer, such as bladder cancer, is screened using a set of at least three chromosomal probes, including those to chromosomes 3, 7, 8, 11, 15, 17, 18, and Y, and wherein aneusomic cells are identified.

Therefore, there is a need in the art to provide methods and compositions that are highly accurate, specific, and sensitive and that produce fewer incorrect diagnoses or undetermined diagnoses. This need is provided by the present invention.

### SUMMARY OF THE INVENTION

Chromosome alterations are characteristic of tumor development and progression, so methods to detect abnormal nuclear DNA content or chromosomal alterations such as DNA cytometry and fluorescence in situ hybridization, for example, are beneficial for sensitive and accurate tumor diagnosis and prognosis. Image cytometry is one method to measure the DNA content of cells of any kind, including urinary cells. An example of this is the AUTOCYTE™ cell analytical system (Carl Zeiss AG; Feldmeilen, Switzerland) using microscopic examination.

On the other hand, FISH is not historically utilized to analyze overall DNA content in a cell nucleus but instead detects numerical or structural anomalies of individual chromosomes. Fluorescent probes targeting particular chromosome regions provide enumeration of chromosome copy numbers, for example, whereas locus-specific probes can identify loss or gain of particular DNA regions. An example of this method is the UroVysion FISH method (Vysis Inc.; Downers Grove, Ill.), which examines cells non-invasively for analysis, wherein the cells are harvested from voided urine. The system detects aneuploidy for chromosomes 3, 7, 17, and loss (deletion) of the 9p21 locus via fluorescence in situ hybridization (FISH) in urine specimens from subjects using four probes labeled with differently colored fluorescent dyes.

The present invention, in contrast, provides a high resolution image analysis in situ cytological method for quantification of telomere length to characterize cells, such as, for example, those in urine from patients with urothelial cancer showing shortened telomeres compared to normal controls. The accuracy of telomere length compared to DNA ploidy, bladder recurrence FISH test, or UBRF (UroVysion, Vysis) and clinical outcome was evaluated, as described herein.

In particular aspects of the invention, there is a method of determining a predisposition to developing cancer in an individual, comprising the steps of: providing a sample from the individual, wherein the sample comprises at least one cell; assaying one or more cells of the sample in situ to determine a telomere length quantity, the quantity comprising a numerical correlation of the mean telomere length and the area of the nucleus; and determining said predisposition of the individual based on the quantity. In specific embodiments, the cell is at least one urothelial cell, at least one bladder cell, or a mixture thereof.

In particular aspects of the invention, the methods of the invention are employed for an individual that is one desired to be tested for a predisposition to developing cancer.

At least some of the methods provided herein concern those that are diagnostic for cancer and/or prognostic for cancer, such as being able to predict relapse for a particular patient, for example. In specific embodiments, the present invention is particularly valuable for cancer diagnosis and prognosis, because it overcomes subjectivity associated with pathological analysis, particularly for samples that are considerably difficult to distinguish (adenomas vs. carcinomas, for example). The present invention provides objectivity for cancer diagnosis by utilizing quantification as a means of cancer diagnosis, thereby circumventing uncertainty upon determination of pathology of cells suspected of being cancerous or of being a particular grade and/or stage of cancer.

In particular, the methods of the present invention employ quantification (as opposed to qualifying, such as by identifying telomeres only as being “shorter” or “longer”) of telomere length in correlation with the area of the nucleus. In specific embodiments, the quantification of the mean telomere length is determined and may be done so by any suitable method, although in preferred embodiments the method is FISH. In other specific embodiments, the quantification of the area of the nucleus is by staining, such as by DAPI staining, although any quantifiable stain would be suitable. In specific embodiments, the staining of the nucleus provides nuclear area, which comprises an integrated optical density as measured by pixel area. It may also be indicative of the chromosomal volume for a cell, such as the substantially total volume of nucleic acid of the cell, such as the double-stranded nucleic acid of the cell. In a particular embodiment, a diagnostic value is identified by comparing the ratio of the average integrated intensity of the area of the nucleus (by stain) over the average area of mean telomere length (by FISH).

It is contemplated herein that the assaying of the telomeres to provide a telomeric quantity may be performed by any suitable methods, although in particular embodiments the quantification comprises in situ hybridization with a polynucleotide that targets telomeric DNA or alternatively by targeting telomeres with an antibody. In specific embodiments, the polynucleotide is labeled, such as, for example, with a fluorophore, a chromagen, or the like. In other embodiments, the antibody is labeled, such as, for example, with a fluorophore, a chromagen, or the like. In another specific embodiment, the methods may also be utilized in an interactive manner, such as upon visual inspection eliminating undesirable and/or irrelevant cells prior to performing the methods.

It is understood by the skilled artisan that the methods described herein may be automated, such as by high throughput analysis. That is, multiple cells may be rapidly screened, and the FISH may be quantified using an algorithm. In a specific embodiment, the methods employ an automated scanning system, such as an online monitoring fluorescence analysis system, including, for example, BioView® (Delta Danish Electronic Light and Acoustics; Venlighedsvej, Denmark).

As described herein in an exemplary embodiment of urothelial cancer (IC), urine specimens from patients diagnosed as positive for UC (6), suspected as having UC (5) or negative or atypical for UC by cytology (8), were evaluated by telomeric DNA FISH staining via digital image analysis software (Universal Imaging Corporation) that measured the average integrated intensity of total nuclear telomeres as a measure of TL. Ten representative cells per slide as (UBRF) were also performed on the same specimens.

The mean TL of patients with positive cytology was 4.83 (range 4.44-5.12), suspicious cytology was 4.5 (range 3.65-4.97), negative or atypical cytology was 5.96 (range 5.40-7.15). Clinical follow up showed that 6/11 patients with TL<5.12 developed within 12 months of the TL test, 4/11 had no follow up at 3 months, and one patient was negative at 18 months. Of eight negative specimens with TL>5.26, none had evidence of UC on follow up (p=0.017). By UBRF and DNA ploidy studies 3/8 negative specimens were abnormal. Bayesian logistic regression showed >99% probability that TL is a better prognostic factor than ploidy or UBRF. The abnormal polysomies and DNA ploidy abnormalities are due to viruses such as the polyma virus, which enter the nuclear DNA and replicate within the nuclei, resulting in false positive cytologies and false aneusomies. TL does not appear to be adversely impacted by this phenomenon.

Thus, shortened telomeres appear to be a most prevalent genetic alteration in UC, and may serve as an accurate indicator of the presence of UC in cytologic specimens. Compared to other available diagnostic adjuncts evaluated herein, shortened TL was more specific than cytology, UBRF and DNA ploidy in predicting the presence of malignancy.

In addition to urothelial cancer, the present inventors conducted TL studies on lung cancer and bronchial brush specimens from ipsilateral to the tumor and contralateral sides. In non-small cell lung cancer, unlike bladder cancer, there generally is an increase in TL in the tumor compared to the ipsilateral bronchial cells, and that TL>5.2 in the tumor predicts overall short survival (less than 24 months) compared to patients whose tumors display shorter telomere length.

The present inventors also evaluated TL in lymphomas and show that longer TL occurs in transformed non-Hodgkin's lymphomas and is associated as well with an overall poor survival.

In one embodiment of the present invention, there is a method of diagnosing and/or prognosticating cancer in an individual, comprising the steps of providing a sample from the individual, wherein the sample comprises at least one cell; assaying one or more cells of the sample in situ to determine a telomere length quantity, such as by FISH; and determining the diagnosis or prognosis of the individual based on the quantity.

The quantity of the telomere length can be defined as a mean value of at least the majority of the telomeres of the cell. In certain aspects, telomere length may be expressed as the average intensity of a signal from the telomeres in a cell or in a collection of cells. In specific embodiments, determining the telomere length quantity comprises assessing a signal indicative of the telomere length. The quantity may be further defined as a numerical correlation of the mean telomere length and the area of the nucleus, such as, for example, a ratio of area of the nucleus to the mean telomere length. The area of the nucleus may be determined by a nuclear stain. Also, the assaying step may be further defined as comprising hybridization of a polynucleotide that targets telomeric DNA or as comprising targeting of an antibody to the telomere.

Although in particular embodiments telomere length is assessed by quantitative FISH methods, in alternative embodiments telomeres are tagged with a telomere-targeting polynucleotide comprising a chromagenic label, such as a biotinylated chromagenic label. A bright field microscope for assaying the stained telomeres tagged with a biotinylated chromagen may employ a similar imaging system for measuring telomere length as indicated by integrated optical density of substantially all telomeres versus total nuclear staining, such as is provided by a hematoxylin counterstain. In particular, this method is more economical than quantitative FISH, since a fluorescent microscope is not required. In yet another alternative, antibodies are utilized to quantitate telomere length, such as by targeting a telomeric protein or by targeting a proteinaceous label comprised on the telomere.

In certain embodiments of the invention, there is a sample comprising one or more abnormal cells. In specific embodiments, the criteria for a sample comprising abnormal cell(s) are as follows: aneusomy of 2 or more chromosomes (3, 7, and/or 17, for example) and/or deletion of 9p21 locus or homozygous loss of 9p21 irrespective of centromeric abnormalities; and/or polysomy or monosomy of 2 or more probes scored as abnormal cells. In some embodiments, if five or more abnormal cells are present, then the specimen is considered positive for cancer.

In specific embodiments, a sample for analysis by methods of the present invention comprises urine, blood, cerebrospinal fluid, pleural fluid, bladder washings, bronchial brush samples, oral washings, touch preps, cheek scrapings, feces, biopsy, fine needle aspirate, nipple aspirates, urine, sputum, bronchiolar alveolar lavage, pap smears, anal scrapings, skin scrapings, or tissue section.

Regarding the ratio of area of the nucleus to the mean telomere length, when the sample comprises at least one bladder cell and the ratio is less than about 5.1, the sample comprises at least one bladder cancer cell. When the sample comprises at least one lung cell and the ratio is greater than about 5.1, the sample comprises at least one lung cancer cell.

The methods of the present invention may be utilized as an initial diagnosis for the individual. They may be utilized for an individual that was previously diagnosed with cancer or an individual that was previously diagnosed as not having cancer. In a specific embodiment, the methods provide the individual with a follow-up diagnosis to a prior non-cancerous diagnosis.

In other specific embodiments, the methods are further defined as utilizing a high throughput analysis for diagnosis and/or prognosis.

In one embodiment of the invention, there is a method of diagnosing and/or prognosticating cancer in an individual, comprising the steps of providing a sample from the individual, wherein the sample comprises at least one cell; assaying one or more cells of the sample in situ to determine a telomere length quantity; and determining the diagnosis or prognosis of the individual based on the quantity. In a specific embodiment, the quantity of the telomere length is a mean value of at least the majority of the telomeres of the cell. In particular aspects of the invention, the determining of the telomere length quantity comprises assessing a signal indicative of the telomere length. In certain aspects, the quantity is further defined as a numerical correlation of the mean telomere length and the area of the nucleus, and the numerical correlation may be further defined as the ratio of area of the nucleus to the mean telomere length. The area of the nucleus may be determined by a nuclear stain, for example.

In specific embodiments, the sample is urine, blood, cerebrospinal fluid, pleural fluid, ascites fluid, bladder washings, bronchial brush samples, oral washings, touch preps, cheek scrapings, feces, biopsy, fine needle aspirate, nipple aspirates, urine, sputum, bronchiolar alveolar lavage, pap smears, anal scrapings, skin scrapings, tissue section, or a mixture thereof.

In particular aspects, the assaying step comprises fluorescence in situ hybridization (FISH). In additional embodiments, when the sample comprises at least one bladder cell and the ratio is less than about 5.1, the sample comprises at least one bladder cancer cell. In other aspects, when the sample comprises at least one lung cell and the ratio is greater than about 5.1, the sample comprises at least one lung cancer cell.

In specific embodiments, the diagnosis of the cancer is an initial diagnosis for the individual. In additional specific embodiments, the individual was previously diagnosed with cancer. In alternative embodiments, the individual was previously diagnosed as not having cancer. Methods of the invention may be further defined as providing the individual with a follow-up diagnosis to the previous non-cancerous diagnosis. In particular, the method may be further defined as utilizing a high throughput analysis for diagnosis and/or prognosis.

In particular aspects, the assaying step is further defined as comprising hybridization of a polynucleotide that targets telomeric DNA. In specific aspects, the polynucleotide comprises a fluorescent label or a chromagenic label.

In another embodiment of the present invention, there is a method of diagnosing low-grade urothelial or bladder cancer in an individual, comprising the steps of providing a sample from the individual, wherein the sample comprises at least one urothelial or bladder cell; assaying one or more cells of the sample in situ to determine a telomere length quantity; and determining said diagnosis or prognosis of the individual based on the quantity. The quantity of the telomere length may be a mean value of at least the majority of the telomeres of the cell. Furthermore, determining the telomere length quantity may comprise assessing a signal indicative of the telomere length. The quantity may be further defined as a numerical correlation of the mean telomere length and the area of the nucleus, and the numerical correlation may be further defined as the ratio of area of the nucleus to the mean telomere length.

In specific embodiments of the invention, the methods are employed to assess telomere length in smokers. It is known that smoking leads to DNA damage and loss of chromosome locus 10q23, which includes the gene for Surfactant A. Deletion of 10q23 has been associated with length of smoking history and is frequently deleted in non-small cell lung cancer, for example. With increased length of exposure to tobacco smoke there is progressive telomere length shortening, which in turn results in formation of dicentric chromosomes, non-reciprocal chromosomal translocations, and genomic instability. Following abrogation of mitotic checkpoints, up-regulation of telomerase (hTERT) occurs, resulting in stabilization of telomere length and cell immortalization. In certain aspects of the invention, these events are associated with the morphologic appearance of non-small cell lung cancer. In further embodiments, shorter telomere lengths were significantly negatively correlated with deletions of 10q23, which in itself is strongly associated with smoking history and poor prognosis. Deletions of 10q23 were also strongly correlated with gene amplification for hTERT. Shorter telomere length was significantly inversely correlated with amplification of hTERT gene, indicating that there is a regulatory feedback pathway between telomere length and gene amplification, in specific embodiments. Similarly, shorter telomeres trended to be associated with higher telomerase expression compared to longer telomeres.

In particular embodiments of the invention, number of pack years of smoking was inversely related to telomere length and was significant at the p=0.5 level. Individuals that never smoked and patients with <16 pack years trended to have longer telomere lengths compared to heavier smokers. In certain embodiments, telomere length had no effect on time to relapse, although in alternative embodiments telomere length does have an effect on time to relapse.

Heavy smoking predisposes to development of non-small cell cancer through short telomere length leading to genomic instability. Once established, non-small cell carcinoma cells are immortalized through maintenance of telomere ends via telomerase. Intracellular telomerase concentration appears to be finely regulated via a negative feedback loop between length of telomeres and gene copy number, with short telomeres leading to gene amplification of hTert on 5p, resulting in increased levels of telomerase. Gene amplification for hTERT is also correlated with loss of surfactant gene. Longer telomeres are associated with lower levels of telomerase expression and absence of 5p gene amplification.

In an additional embodiment of the present invention, there is a kit for determining a diagnosis and/or a prognosis of cancer for an individual, housed in a suitable container and comprising one or more of the following: one or more telomere-targeting molecules; a label; and a nuclear stain. The one or more telomere-targeting molecules comprises a polynucleotide that targets telomeric DNA. The label may comprise a fluorophore, for example. The one or more telomere-targeting molecules may comprise an antibody. The label may comprise a chromagen, for example. The kit may further comprise instructions for the kit, wherein the instructions comprise an expected ratio of nuclear area to quantity of telomere length, wherein the ratio isindicative of said cancer. The kit may further comprise a sample collector.

In specific embodiments, the fluorophore is 7-aminomethylcoumarin-3-acetic acid (AMCA), 5- (and -6)-carboxy-X-rhodamine, lissamine rhodamine B, 5-carboxyfluorescein, 6-carboxyfluorescein, fluorescein-5-isothiocyanate (FITC), 7-diethylaminocoumarin-3-carboxylic acid, tetramethylrhodamine-5-isothiocyanate, tetramethylrhodamine-6-isothiocyanate, 5-carboxytetramethylrhodamine, 6-carboxytetramethylrhodamine, 7-hydroxycoumarin-3-carboxylic acid, 6-[fluorescein 5-carboxamido]hexanoic acid, 6-[fluorescein 6-carboxamido]hexanoic acid, N-(4,4-difluoro-5,7-dimethyl-4-bora-3a,4a diaza-3-indacenepropionic acid, eosin-5-isothiocyanate, or erythrosin-5-isothiocyanate. The nuclear stain may be DAPI, Hoechst 33342 dye, 7-actinomycin-D/7-Aminoactinomycin D/Chromomycin A3, propidium iodide, or Nuclear fast red. The sample collector may be a cup, a toothpick, a loop, a syringe, a bronchial brush, a needle, a cotton swab, or a cyto brush.

In additional embodiments of the invention, the methods of the invention are employed to monitor response to treatment of a cancer therapy. For example, telomere length/nuclear area assessment may be employed prior to a cancer therapy, and following one or more rounds of the cancer therapy the telomere length/nuclear area assessment may be evaluated. If the assessment indicates that the therapy is not successful, then an alternative therapy may be employed.

In another embodiment, there is a method of differentiating a cell having a polyoma virus infection from another cell that does not have a polyoma virus infection, comprising the steps of: providing at least one cell suspected of having a polyoma virus infection; assaying one or more cells of the sample in situ to determine a telomere length quantity; and determining whether or not the cell has a polyoma virus infection based on the quantity.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 illustrates cytology of a normal bladder control cells upon analysis by methods of the present invention.

FIG. 2 provides cytology of bladder cells infected with polyoma virus using methods of the present invention.

FIG. 3 shows cytology of bladder cells infected with polyoma virus using methods of the present invention.

FIG. 4 demonstrates cytology of atypical bladder cells c/w high grade transitional cell carcinomas identified by methods of the present invention.

FIG. 5 demonstrates cytology of transitional cell carcinoma as identified by methods of the present invention.

FIGS. 6A-6D show different cytological diagnosis with the telomere FISH staining method of the present invention.

FIG. 7 shows telomere length as a function of the average intensity of the telomeres as determined by the present invention.

FIGS. 8A-8C provide representative assayed images of normal bronchial brush (FIG. 8A), tumor bronchial brush (FIG. 8B), and tumor touch preps (FIG. 8C).

FIGS. 9A-9D show representative assayed images (FIGS. 9A and 9B) and corresponding exemplary linescans for normal bronchial brush and tumor touch preps (FIGS. 9C and 9D).

FIGS. 10 and 11 show hTERT images showing amplification of the hTERT gene (shows as green in a color photo) in comparison to the centromeric chromosome 5 (shows as red in a color photo).

FIG. 12 demonstrates telomere length in different subtypes of lymphoma.

FIG. 13 shows telomere length for different grades of follicular lymphoma.

FIG. 14 shows telomere length correlated with age and lymphoma subtypes.

### DETAILED DESCRIPTION OF THE INVENTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “bladder cancer” as used herein refers to cancer of the bladder.

The term “bladder sample” as used herein refers to a sample from an individual wherein the sample is indicative of the state of at least one bladder cell of the individual. For example, the bladder sample may comprise urine, such as is voided or obtained by catheter; one or more cells, which may be obtained by catheter or through a biopsy, for example; or a mixture thereof. In a preferred embodiment, the bladder sample comprises urine having one or more bladder cells sloughed from the bladder and is obtained by non-invasive means, such as by voiding.

The term “cytologically” as used herein refers to of or relating to the formation, structure, and function of cells.

The term “in situ” as used herein refers to an original or natural place or site. Regarding specific aspects of the invention, in situ hybridization includes the steps of fixing a biological sample, hybridizing a chromosomal probe to target DNA contained within the fixed biological sample, washing to remove non-specific probe binding, and detecting the hybridized probe.

The term “low-grade” bladder or urothelial cancer is defined as a tumor that is very well-differentiated and resembles the normal bladder mucosa. It is usually papillary and has an indolent biologic behavior.

The term “lung sample” as used herein refers to a sample from an individual wherein the sample is indicative of the state of at least one lung cell of the individual. For example, the lung sample may comprise sputum; one or more cells, which may be obtained by biopsy, for example; or a mixture thereof. In a preferred embodiment, the lung sample comprises sputum having one or more lung cells sloughed from the lung and is obtained by non-invasive means, such as expectorating the sputum.

The term “lymphoma sample” as used herein refers to a sample from an individual wherein the sample is indicative of the state of at least one lymph cell of the individual. For example, the lymphoma sample may comprise fine needle aspirate; one or more cells, which may be obtained by biopsy, for example; or a mixture thereof.

The term “quantity of telomere length” as used herein refers to an amount indicative of the length of the telomere and does not refer to the qualitative assessment of telomeres being either “short” or “long,” for example. In a specific embodiment, the amount is not an absolute numerical quantity of the exact number of telomeres in a particular cell but is representative of the length of the telomeres, and in particular embodiments it is representative of the mean length of the telomeres.

The term “urothelial cancer” as used herein refers to cancer of the layer of transitional epithelium in the wall of the bladder, ureter, and renal pelvis, external to the lamina propria.

### II. The Present Invention

The present invention provides methods and compositions useful for diagnosing, prognosticating, or both, of cancer. Any type of cancer may be suitable to the present invention. In particular, the invention regards quantifying telomere length as a means of determining a predisposition to developing cancer, diagnosing cancer, prognosticating cancer, or both. It is known that normal somatic cells lack telomerase and that telomeres shorten with cell cycle division due to their incomplete replication; when telomeres shorten to a critical length, cell senescence occurs. Tumorigenesis is associated with shortened telomeres leading to telomere dysfunction, chromosomal instability, and upregulation of telomerase leading to stabilization of chromosomes. Maintenance of telomere length is required to acquire replicative immortality, and this occurs through the activation of telomerase. Human telomerase reverse transcriptase gene (hTERT) is encoded by the hTERT gene on chromosome 5p15.33, which is a determinant for telomerase activity control.

Specifically, the invention regards comparing values indicative of the nuclear area and mean telomere length for at least one particular cancer cell, for at least one cell suspected of being cancerous, or for at least one cell for establishing a baseline value prior to an individual being suspected of having cancer.

For the exemplary embodiment concerning urothelial or bladder cancer, urinary cytology is often used in combination with cystoscopy both for primary bladder cancer diagnosis and for monitoring recurrance, particularly after transurethral resection. Urinary cytology is very specific for poorly differentiated urothelial carcinoma detection (Grade 3), but provides considerably unreliable specificity for low-grade tumors. To complicate the process, there is much cytological overlap between urothelial change and low-grade urothelial neoplasia, so many samples are therefore diagnosed as cellularly atypical. Furthermore, many false-positives and poor reproducibility plague current cytological methods. Thus, better diagnostic and prognostic methods are warranted.

Chromosomal alterations are likely tumor-specific, and they occur frequently in cancers, such as urothelial or bladder cancer. Therefore, methods to characterize chromosome abnormalities are useful. In some embodiments, methods are provided that employ qualitative analysis of chromosome abnormalities (e.g. aneuploid vs. diploid; short telomeres vs. long telomeres), although the present invention provides a unique and highly accurate quantitative analysis of chromosome alterations, particularly at the telomeres.

Although a variety of methods for providing diagnoses of bladder cancer are currently known, in some cases a combination of tests must be utilized in order to provide a correct diagnosis. For at least some individuals suspected of having cancer, sensitive diagnostic tests are required for accuracy, such as those that detect chromosomal differences upon the change from normal cell to a cancer cell, including by in situ hybridization (Katz et al., 1997).

This invention provides an in situ method to quantify telomere length on a per cell basis in clinical cytology specimens. Total telomere fluorescence (such as through FITC signals) is calculated as mean telomere length based on the area of the nucleus, as measured by, for example, a DAPI counterstain. Based on the findings in the exemplary lung cancer and bladder cancer, TL measurement is a powerful tool for both diagnosis and prognosis of carcinoma. That is, in patients with established lung cancer, the ratio of TL of bronchial brush cells on the ipsilateral side compared to the TL of bronchial brush cells on the contralateral side predicted the presence of lung cancer. In lung cancers, TL>5.2 predicted poor prognosis. In bladder cancer, shortened telomere length was a powerful predictor of the presence of cancer versus other causes of abnormal DNA content, such as viral infection.

Presently, the newly introduced 4-color UroVysion FISH test by Vysis is the current state of the art test for diagnosing urothelial cancer in cytology specimens. The present invention is superior in that it avoids false negative diagnosis and, instead of a 4 color probe, it only utilizes a single labeled anti-telomeric probe (such as one labeled with FITC), thus avoiding the need for expensive filter wheels. In addition, the commercial probe is extremely cheap compared to the VYSIS probe and the time taken for probe staining is shorter. The speed of results with the present invention probe analysis could be increased with software other than the exemplary Metamorph Offline for quantification of TL, in specific embodiments. In addition, some existing technology uses a Southern Blot and chemiluminescence technique (Teloquant assay kit, Pharmingen; San Diego, Calif.), which can take up to three days to prepare. It also may introduce contaminating normal tissue, as it is not done on a per cell basis. Another method of quantifying TL relies on a Laser Cytometer, a very expensive instrument. The method described herein is practical, as a relatively inexpensive software program can be hooked up to an existing PC.

### III. Bladder Cancer

Although the methods and compositions of the present invention are applicable to any form of cancer, in one specific embodiment it is useful for bladder cancer. The bladder is a hollow, balloon-like organ lying in the pelvis, which collects urine from the kidneys via tubes called ureters and stores it until it is full enough to empty through the urethra. Cancer of the bladder comprises uncontrolled growth of bladder cells and is more common in men than in women. Although the cause is unknown, smoking and certain chemicals may be related.

The most common symptom of bladder cancer is haematuria, which is blood in the urine. Haematuria may appear suddenly with no apparent cause, and often there is no pain associated with it, although sometimes blood clots can form and cause pain or obstruction to the flow of urine. The presence of haematuria may come and go. The color of the urine during haematuria may vary from rusty brown to deep red, depending on the amount of blood, and the amount of blood is not related to the extent of the cancer. Other symptoms include dysuria (difficult or painful urine discharge); urinary frequency or urgency; flank pain secondary to obstruction; and pain from pelvic invasion or bone metastases.

There are different types of bladder cancers. Most bladder cancers are termed superficial, and resemble tiny polyps in appearance, growing on the inside lining of the bladder; they can be single or multiple. They are sometimes referred to as papillary tumors, papillomas, or bladder warts. They can be removed by surgical excision and cauterization to prevent bleeding. In addition to the cystoscopic removal of the tumor and regular cytoscopies, intravesical chemotherapy may be performed, wherein washing of the bladder is performed regularly with one or more of several chemotherapeutic drugs. This treatment is usually given on a weekly basis for about 6-8 weeks.

If the bladder cancer has grown deeper into the bladder wall and extends into the muscle layer or its surrounding tissues it is described as invasive, in which case surgery, radiotherapy and chemotherapy may be used alone or in combination to treat invasive bladder cancer. Surgeries for invasive bladder cancer may include transurethral resection of a tumor, which is referred to as partial cystectomy. That is, if the tumor is confined to the bladder wall, it may be possible to remove the tumor and only the section of the bladder involved. This may be performed either as a telescopic procedure (cystoscopic resection) or as a cutting operation through the abdomen (partial cystectomy). In other cases, such as when the tumor is more extensive, there may be total removal of the bladder (referred to as a complete or radical cystectomy. In any case, recurrence of the tumor following resection occurs frequently. Chemotherapeutics include M-VAC (methotrexate, vinblastine, adriamycin (doxorubicin), and cis-platinum).

In cases wherein the bladder cancer has spread to the lymph nodes, bone, lungs or sites other than the bladder, this is termed metastatic bladder cancer. In these cases, chemotherapeutics may be employed, such as M-VAC, paclitaxel, and gallium nitrate with vinblastine and ifosfamide, for example.

In particular embodiments, radiotherapy treats the cancer, such as with high energy x-rays. It may be given before, after or instead of surgery, depending on the particular individual.

Chemotherapy may also be administered, and in some cases is done so in addition to some form of surgery or radiotherapy rather than on its own. In particular embodiments, some of the chemotherapy may be provided via intravenous infusion, by injection, or by other methods.

### IV. Current Diagnostic Procedures

There is provided herein methods and compositions related to cancer diagnostics and prognostics. In particular embodiments, any cancer diagnosis and/or prognosis may be identified by these methods, although the exemplary bladder, urothelial, lung, and lymphoma cancers are demonstrated herein.

The methods are suitable for a subject in need of an evaluation for the presence and/or prognosis of cancer. A “subject in need of evaluation” includes any subject who may reasonably be tested for the presence of bladder cancer, in exemplary embodiments, including, but not limited to, a subject who exhibits at least one sign of bladder cancer, such as hematuria; difficult, unduly frequent and/or painfull urination; and/or being at risk for developing bladder cancer. Subjects at risk for developing bladder cancer include those subjects having a history of bladder cancer or toxin exposure, subjects having indwelling urinary catheters, smokers, and patients suffering from or having a history of Schistosomiasis infection. Other subjects in need of such evaluation are subjects who have been previously diagnosed and treated for bladder cancer and who need follow-up evaluation for recurrent disease. Alternatively, a “subject in need of such evaluation,’ may be asymptomatic and may merit evaluation only for routine screening purposes.

Generally, some of the methods currently utilized for the exemplary bladder cancer detection include the detection of tumor antigens present on the tumor surface that are also present in urine; detection of abnormal blood group antigen expression; detection of growth factors and receptors from tumors; detection of tumor enzymes, such as telomerase; detection of protein fragments from tumor activity; detection of chromosomal abnormalities; detection of chromosomal abnormalities in voided urine cell samples; detection of tumor mRNA from RT-PCR; and microsatellite analysis of sediment from urinary DNA (Little, 2003). Although there are a variety of presently known diagnostic procedures related to the exemplary cancer, bladder cancer, these procedures are insufficient compared to the present invention. In alternative embodiments, one or more of these methods may be used in conjunction with methods of the present invention.

A. Urinalysis/Cytology

Although urine cultures often fail to detect malignancy, other procedures exist to assist in the diagnosis, such as cytological studies. However, these tests are less than accurate, and due to a rather high rate of false negatives, cannot exclude the possibility of malignancy. The tests in use are less accurate in detecting low grade transitional cell carcinoma (TCC), in particular. Other different types of tests used in bladder cancer diagnosis encompass urine markers, such as NMP22, BTA, FISH and others.

B. Cystoscopy

In cystoscopy, a cystoscope is inserted into the urethra and up into the bladder. Any noteworthy characteristics of the bladder are photographically recorded through a thin-lighted tube, noting any abnormalities and where they are located. A flexible cystoscope may be used for surveillance, while a rigid cystoscope may be used to remove (or biopsy) tissues. Cystoscopy is historically the most reliable tool used in diagnosing the presence of tumors.

C. Photodynamic (Fluorescence) Cystoscopy—Hexvix®

Approved in Europe, PhotoCure ASA's multicenter European phase III study reported that Hexvix® fluorescence cystoscopy identifies approximately 30% more patients with aggressive bladder cancer (carcinoma in situ) compared to standard cystoscopy.

In this method, a solution is provided into the bladder and held for one hour before the fluorescent light cystoscopy is performed. In aspects of this method, a bladder wash may be employed. That is, a saline solution is administered through the cystoscope, and the bladder is vigorously irrigated, which loosens cells from the lining of the bladder. Upon biopsy and if abnormal tissue is found, the doctor may obtain the sample and request pathology of at least part of the sample. After surgical removal through the scope, tissues are cauterized to lessen bleeding and hasten healing. Biopsy is the most reliable procedure for the diagnosing of CIS and/or TCC of the bladder, prior to this invention.

D. Ultrasound (US)

Ultrasonography (ultrasound) uses sound waves for imaging, which may be recorded on x-ray. The image of the internal organ may provide information of bladder malignancy.

E. Intravenous Pyelography (IVP).

An IVP involves an intravenous injection of contrast material, which is then filtered out of the blood in into the urine by the kidney. Standard x-rays taken during this process show the urinary tract. This test is particularly useful for visualizing the upper tract.

F. Retrograde Pyelography

Like the IVP, this test uses a special dye to outline the lining of the bladder, ureters, and kidneys on x-rays, although with retrograde pyelography the dye is injected through a urinary catheter rather than into a vein.

G. Computed Tomography (CT)

The CT scan is commonly used as a diagnostic tool for staging and follow-up. Often a contrast-medium is additionally injected into a vein to assist the visualization. A CT scan of the pelvis will provide information about whether the cancer may have spread to tissues next to the bladder, to nearby lymph nodes in the pelvis, or to distant organs such as the liver. CT scans are used primarily if spread beyond the bladder is suspected. Sometimes, a MRI scan is used instead of the CT scan.

H. MRI

Magnetic resonance imaging is similar to CT scans but uses powerful magnets and radio waves instead of x-rays to take detailed cross-sectional images. If spread beyond the bladder is suspected, MRI scans are sometimes used to detect cancer in tissues next to the bladder, in nearby lymph nodes, or in distant organs.

I. MR Lymphography

MR lymphography is a new and promising imaging modality in differentiating benign and metastatic lymph nodes, which gives information on both lymph node morphology and function.

J. Transurethral Resection (TUR)

Transurethral resection is a minimally invasive surgical technique where tumors are removed through the urethra via a scope equipped with a special tool on the end for excision of tissue. Cauterization prevents excessive bleeding.

K. Electrosurgery/Laser Surgery

Electrosurgery uses an electric current to remove the cancer. The tumor and the area around it are burned away and then removed with a sharp tool.

Laser therapy uses a narrow beam of intense light to remove cancer cells. Laser surgery is often used to destroy small low-grade tumors and is performed through a cystoscope.

L. Pathology Tests

Often a bladder cancer patient will be told that tissues have not had extensive pathology testing; this may be because the tumor was obviously superficial and the cells well differentiated. Laser or cauterization techniques may rule out path tests as small tumors and tissues are destroyed during removal, thus a doctor may be relying on his experience. The common approach to superficial tumors is TUR followed by continued (such as quarterly or bi-annual) surveillence. The research shows that this is almost always a safe approach, since with the large majority of these cases true (biological) progression is rare, occurring less than 5% of the time; thus, in the case of superficial papillary tumors with well-differentiated cells, extensive pathology testing as well as aggressive treatment may be reserved for multiple recurrences or presence of other risks factors.

In the case of carcinoma in situ, multiple tumors and multiple tumors of mixed cellular origin, or at any evidence of subepithelial invasion (stage T1), resected bladder tumors should always be submitted for pathological testing in order to determine the pT (post surgical stage) category.

A tumor is staged as pTx if there is insufficient or inadequate material available to the pathologist for a proper assessment of invasion. Since it is frequently not possible to determine whether or not invasion has occurred, a pTx tumor may be entirely superficial and non-invasive. The text of the pathology report should state clearly whether or not invasion has been identified in the material examined. It is generally not possible to differentiate between superficial and deep detrusor muscle in biopsy samples, and a cystectomy specimen is necessary before a pathologist can reliably subdivide muscle invasive tumors into pT2 or pT3 categories.

### V. Staging/Grading of Cancer

Although in particular embodiments any type of cancer may be staged or graded with the methods and compositions of the present invention, in a specific embodiment the present invention is useful for staging/grading bladder cancer.

The stage refers to how far a cancer has progressed anatomically, while the grade refers to cell appearance (differentiation) and DNA make-up. Stage is determined by the depth to which the tumor has penetrated the bladder wall, and assessment of invasion of lymph nodes and other surrounding organs and tissues. The grade is determined by pathology tests, showing how abnormal or aggressive the cells of biopsy specimens appear, and how closely a tumor resembles normal tissue of its same type. Differentiation is another term used to describe the degree of an abnormal cell's resemblance to its normal counterpart. Tumor cells are described as well-differentiated when they look much like normal cells of the same type and are able to carry out some functions of normal cells. Poorly differentiated and undifferentiated tumor cells are disorganized and abnormal-looking. As a general rule, the grade of a tumor corresponds to its rate of growth or aggressiveness. An undifferentiated or high-grade tumor grows more quickly than a well-differentiated or a low-grade one. A large tumor can be low-grade, and a small tumor can be high grade. Although TIS (also written as CIS—carcinoma in situ) presents as superficial, carcinoma in situ is a potentially dangerous and usually high-grade tumor, and CIS patients are at greater risk for progression and must be monitored closely.

There are multiple classification systems to classify bladder tumors. For example, the World Health Organization (WHO) classification recognizes three grades of urothelial carcinoma. Grade 1 represents well-differentiated papillary tumors with limited atypia and mitoses. Grade 2 represents a bladder tumor with more cytological atypia and mitoses than Grade 1, but less than Grade 3. At the other end, Grade 3 lesions show a marked increase in the cell layers and cell size, and noticeable pleomorphism and mitoses are prominent. Tumor grade appears to correlate significantly with the natural history of transitional cell carcinoma. The higher the grade of the diagnosis, the higher the incidence of death from the disease within two years.

In an alternative embodiment, two staging systems for bladder cancer other than that of the WHO are utilized: the American Joint Committee on Cancer/International Union Against Cancer Tumor-Node-Metastasis (TNM) system and the Jewett-Marshall staging system. The two systems are compared in Table 1.

TABLE 1
Comparison Between Jewett-Marshall
and TNM Staging

Staging system

| Jewett- | | |
|---|---|---|
| Marshall | TNM | Description |
| O | T0 | No definitive tumor |
| | Tis | Carcinoma in situ |
| A | Ta | Papillary tumor without |
| | | invasion |
| | T1 | Lamina propria invasion |
| B-1 | T2 | Superficial muscle invasion |
| B-2 | T3a | Deep muscle invasion |
| C | T3b | Perivesical fat invasion |
| D-1 | T4 | Prostate, vagina, uterus, or |
| | | pelvis side wall invasion |
| D-2 | N1-3 | Pelvic lymph-node metastasis |
| D-3 | M | Lymph-node metastasis |
| | | beyond pelvis |
| D-4 | M | Distant metastasis |

TNM = tumor-node-metastasis

Table 2 summarizes the nodal classifications utilized in the TNM system.

TABLE 2
Nodal Classification (TNM Staging
System)

| Stage | Description |
|---|---|
| N1 | Metastasis in single node, <2 cm |
| N2 | Metastasis in bilateral lymph nodes, or |
| | single node >2 cm but <5 cm |
| N3 | Metastasis in any node >5 cm |
| M | Lymph-node metastasis beyond pelvis, |
| | or distant metastasis |

TNM = tumor-node-metastasis

Although any suitable system may be utilized to determine the staging and/or grading of bladder cancer, the present invention is particularly well-suited to substituting or supplementing the aforementioned systems by providing a quantitative representation of telomere length for characterizing grade and stage.

Bimanual examination in order to detect palpable masses is another important part of clinical staging. The presence of a mass palpable on bimanual examination is of prognostic value and incorporation of this feature with microscopic tumor invasion may enhance the usefulness of clinical staging.

Pathology tests can also be done that analyze various biomarkers/prognostic indicators. These findings combined with results of all diagnostic procedures performed can help to best define treatment strategies. Biopsy alone cannot always accurately assess the depth of invasion, thus the grade as determined in the path lab is an integral part of staging.

Many different factors can effect the course of treatment, and every case is unique; the extent of tumor invasion, large or multi focal tumors, ureter obstruction, rare histological cell type, carcinoma in situ, compromised renal function are important prognosis factors. If a person is not a good surgical candidate, or has concomittant medical problems, this may also substantially influence which treatment strategy is recommended as well as the prognosis.

Out of all patients with bladder cancer, about 50% belong to the low-risk group, 35% to the intermediate group, and 15% to the high-risk group. Patients belong to the low-risk group if they have a single primary or recurrent Ta grade 1 or Ta grade 2 lesion, or the high-risk group if they have multiple primary or recurrent T1 grade 3 lesions and/or if the tumor(s) are larger than 3 cm. In between there are patients with multiple but less than seven Ta grade 1 or Ta grade 2 lesions: they have an intermediate prognosis.

Clinical staging, including nuclear imaging, often underestimates the extent of tumor invasion, particularly in cancers that are less differentiated and more deeply invasive. In a study that reviewed accuracy of staging in 130 cystectomy patients, the overall clinical staging error was 61.5%, with 41.5% of the cancers understaged. Of the patients with Carcinoma in situ, 60% were found to be of greater extent than pT1 tumors. The authors stated that clinical errors in classification are common and impair the evaluation of neoadjuvant treatments. This supports an aggressive approach when these patients do not respond promptly to intravesical chemotherapy (Soloway et al., 1994).

Although cystoscopy is a very reliable follow up tool, it also has a small margin of error. Unfortunately there is no currently available reliable test which is accurate enough to detect microscopic metastases, until the present invention.

### VI. Sample Collection and Preparation

The present invention encompasses obtaining a sample from an individual known to have cancer, suspected of having cancer, or suspected of being susceptible to getting cancer. In one embodiment, a sample is collected from the individual to provide at least one cell for analysis with methods as described herein.

In a particular embodiment, bladder cancer cells are obtained from a urine sample. Exfoliated cells from the sample may be collected, undesirable cells (such as red blood cells, white blood cells, etc.) and material (such as necrotic tissue or cellular debris) may be removed from the sample prior to analysis.

A urine sample, according to the invention, may be a voided urine sample or may be obtained by catheterization. In preferred, non-limiting embodiments, the volume of the urine sample is at least 20 ml, and more preferably at least 100 ml. In preferred, nonlimiting embodiments of the invention, the sample size is such that at least 50-400 exfoliated cells, and more preferably at least 200 exfoliated cells, are present in the sample. The term “exfoliated cell” refers to a normal or malignant cell having its origin in the mucosa of the bladder.

In specific embodiments, samples are collected in accordance with the substantially non-invasive methods provided in U.S. Pat. No. 6,054,314. As described therein, energy from an external source is applied to the subject such that it is sufficient to loosen cells from the internal surface of an internal organ so that at least some of the loosened cells are detached from the internal cellular surface of the organ. In specific embodiments, the internal organ is a bladder, colon, kidney, prostate, uterus, stomach, pancreas, or lung. In a more specific embodiment, bladder epithelial cells are collected by this method. A cancer disease state may be identified subsequent to this process, such as by identifying telomerase expression.

Other samples for collection, which may be obtained by standard methods in the art, include blood, cerebrospinal fluid, pleural fluid, bladder washings, bronchial brush samples, oral washings, touch preps, cheek scrapings, feces, biopsy, fine needle aspirate, nipple aspirates, urine, sputum, bronchiolar alveolar lavage, pap smears, anal scrapings and skin scrapings, and so forth.

Typically, cells are harvested from a biological sample using standard techniques. For example, cells can be harvested by centrifuging a biological sample such as urine, and resuspending the pelleted cells. Typically, the cells are resuspended in phosphate-buffered saline (PBS). After centrifuging the cell suspension to obtain a cell pellet, the cells can be fixed, for example, in acid alcohol solutions, acid acetone solutions, or aldehydes such as formaldehyde, paraformaldehyde, and glutaraldehyde. For example, a fixative containing methanol and glacial acetic acid in a 3:1 ratio, respectively, can be used as a fixative. A neutral buffered formalin solution also can be used, and includes approximately 1% to 10% of 37-40% formaldehyde in an aqueous solution of sodium phosphate. Slides containing the cells can be prepared by removing a majority of the fixative, leaving the concentrated cells suspended in only a portion of the solution.

The cell suspension may be applied to slides such that the cells do not overlap on the slide. Cell density can be measured by a light or phase contrast microscope. For example, cells harvested from a 20 to 100 ml urine sample typically may be resuspended in a final volume of about 100 to 200 μl of fixative. Three volumes of this suspension (usually 3, 10, and 30 μl), are then dropped into 6 mm wells of a slide. The cellularity (i.e. density of cells) in these wells is then assessed with a phase contrast microscope. If the well contains a volume of cell suspension that does not have enough cells, the cell suspension is concentrated and placed in another well.

Prior to in situ hybridization, chromosomal probes and chromosomal DNA contained within the cell each may be denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3M sodium chloride and 0.03M sodium citrate). Denaturation conditions typically are established such that cell morphology is preserved. Chromosomal probes can be denatured by heat. For example, probes can be heated to about 73° C. for about five minutes.

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations are typically performed in hybridization buffer containing 1-2×SSC, 50% formamide and blocking DNA to suppress non-specific hybridization. In general, hybridization conditions, as described above, include temperatures of about 25° C. to about 55° C., and incubation lengths of about 0.5 hours to about 96 hours. More particularly, hybridization can be performed at about 32° C. to about 40° C. for about 2 to about 16 hours.

Non-specific binding of chromosomal probes to DNA outside of the target region can be removed by a series of washes. Temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

### VII. Fluorescence In Situ Hybridization (FISH)

Fluorescence in situ hybridization (FISH) is utilized in particular methods of the present invention. FISH uses fluorescent molecules to vividly localize or identify genes or chromosomes. This technique is particularly useful for gene mapping and for identifying chromosomal abnormalities.

FISH utilizes short sequences of single-stranded DNA, called probes, that are complementary to the desired DNA sequence. These probes hybridize, or bind, to the complementary DNA and, because they are labeled with fluorescent tags, allow an individual to see the location of those sequences of DNA. Unlike most other techniques used to study chromosomes, which require that the cells be actively dividing, FISH can also be performed on nondividing cells, and it is therefore a highly versatile procedure.

FISH probes usually fall into one of three categories, including: locus-specific probes, which hybridize to a particular region, such as a particular gene, of a chromosome; alphoid or centromeric repeat probes generated from repetitive sequences found at the centromeres of chromosomes; or whole chromosome probes, which are actually collections of smaller probes, each of which hybridizes to a different sequence along the length of the same chromosome. In the present invention, telomeric-specific FISH probes are utilized to identify substantially only the telomeres of one or more chromosomes.

### VIII. Telomeric Probes

The methods of the present invention employ probes specific for the telomere, in particular embodiments. In particular, the telomere probe identifies one or more specific sequences indicative of the telomeres. In specific embodiments, the probe targets the 5′-TTAGGG-3′ sequence that is highly repetitive at the telomeres.

In specific embodiments, fluorescent telomere probes are utilized. Fluorophores of different colors may be chosen such that the telomeric probe can be distinctly visualized. For example, one of the following fluorophores may be used: 7-amino-4-methylcoumarin-3-acetic acid (AMCA), Texas Red™ (Molecular Probes, Inc., Eugene, Oreg.), 5- (and -6)-carboxy-X-rhodamine, lissamine rhodamine B, 5- (and -6)-carboxyfluorescein, fluorescein-5-isothiocyanate (FITC), 7-diethylaminocoumarin-3-carboxylic acid, tetramethylrhodamine-5- (and -6)-isothiocyanate, 5- (and -6)-carboxytetramethylrhodamine, 7-hydroxycoumarin-3-carboxylic acid, 6-[fluorescein 5- (and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc., Eugene, Oreg.). Probes are viewed with a fluorescence microscope and an appropriate filter is utilized for the fluorophore.

Probes also can be indirectly labeled with biotin or digoxygenin, or labeled with radioactive isotopes such as ³²P and ³H, although secondary detection molecules or further processing then is required to visualize the probes. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard calorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

### IX. Nuclear Stain

The present invention comprises determining the area of the nucleus in determination of a ratio of nuclear area to telomere quantity as indicative of diagnosis and/or prognosis of cancer. In specific embodiments, a stain is utilized to determine the nuclear parameter of the ratio. Any suitable nuclear stain that can be quantified may be utilized, although in specific embodiments the nuclear stain is fluorescent or chromatogenic.

Exemplary nuclear stains include, for example, DAPI, Hoechst 33342 dye, 7-actinomycin-D/7-Aminoactinomycin D/Chromomycin A3, propidium iodide, or Nuclear fast red. In specific embodiments, DAPI is employed for nuclear staining. It is known that DAPI (4′,6-diamidino-2-phenylindole) is a stain that is used to stain nucleic acid, such as double stranded DNA. It is a colorful stain having blue fluorescence that attaches to the minor groove of the DNA helix around A-T clusters.

### X. Kits of the Invention

The diagnostic/prognostic methods and compositions of the invention are particularly well-suited for providing kits. Kits of the invention may comprise one or more means for collection of samples, such as a cup for sputum or urine, bronchial brush etc., a toothpick, a loop, a syringe, a bronchial brush, a cyto-brush for papsmears, cotton swab, fixatives for collection include Ringer's lactate, Saccomano's fixative, 50% alcohol, RPMI-1640; the telomeric probe, such as the telomere-targeting DNA and/or the fluorophore or a biotinylated chromagen tagged to the telomeric probe to be used with a light hematoxylin counterstain; nuclear stain, such as DAPI, Hoechst 33342 dye, 7-actinomycin-D/7-Aminoactinomycin D/Chromomycin A3, propidium iodide, or Nuclear fast red, for example; and/or instructions for utilizing the kit, to name a few.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Fluorescence In Situ Hybridization for Telomeres

Slides were pretreated in 2× sodium saline citrate (SSC) for 2 minutes at 73° C. Slides were then digested with 0.5 mg/ml protease (Vysis Inc., Downers Grove Ill.) in 1×PBS, pH 2.0 at 37° C. for 8 minutes, washed with water and rinsed in 1×PBS for 5 minutes, fixed in 1% formaldehyde in 1×PBS and again rinsed in 1×PBS for 5 minutes. Slides were then denatured with 70% formamide in 2×SSC at 74° C. for 5 minutes and quenched with cold 70% ethanol for 2 minutes, then dehydrated and air-dried. The PNA telomere probe mixture (Applied Biosystems, MA) was denatured at 74° C. for 5 minutes, applied to denatured slide, coverslipped, sealed with rubber cement and incubated in a humid chamber at room temperature for hybridization. After hybridization for 4 hours, slides were washed at 57° C. in 0.1% Tween 20 in 1×PBS for 30 minutes and then rinsed in 0.1% Tween 20 in 2×SSC for 1 minute at room temperature, and air-dried. Slides were then counterstained with 10 μl of 10 μg/ml 4,6-diaminidino-2-phenylidole (DAPI) in Vectashield mounting medium (Vector Laboratories) and coverslipped.

### Example 2

### Image Capturing

The images are captured by fluorescent microscope (Leica DMLB) equipped with 100-watt mercury lamp and Vysis filter set DAPI single band pass (DAPI counterstain), and Green single band pass at 63×. Ten (urine) or fifty (bronchial) non-overlapping cells and nuclei with distinct signals were captured using the same exposure, gain and offset (Exposure=1 second, Gain=50, Offset=0). These images were then converted into 8-bit grey scale TIFF files and are then analyzed with Metamorph Offline Software (Universal Imaging Corporation, PA). The software measured the average integrated intensity of total nuclear telomeres as a measure of telomere length.

### Example 3

### Telomere Length in Bladder Cancer

Telomere length was assessed in individuals known to have bladder cancer, suspected of having bladder cancer, or not having bladder cancer. Table 3 provides data concerning the measurements for the average integrated intensity (the area of the nucleus) over the average area (the intensity of the telomeres), which determines the average intensity of TL.

The column entitled “Abnormal Cells” refers to the number of cells that were considered abnormal by UroVysion FISH analysis standards (wherein a cell is classified as abnormal when the ploidy is greater or less than diploid in two or more chromosomes or 9p21. It is noteworthy that the individuals with patient ID numbers 9, 10, and 11, for example, were classified as normal by UroVysion FISH, whereas the methods of the present invention classified them as having cancer (based on average intensities of 4.5, 4.55, and 4.97, respectively), and upon follow-up two of three individuals were diagnosed as having cancer by multiple methods. The third patient had a carcinoma of the bladder resected one week before the telomere length test. He subsequently received BCG therapy and 15 months later has not had recurrence.

| | | | | DATE | | | | | | | | | LAST | |
|---|---|---|---|---|---|---|---|---|---|---|---|---|---|---|
| | | | | CYTOL. | | | | | | | | | FOLLOW- | |
| | | AVG INTEGR. | | UROV. | | | | ABN. | | Cells > | DATE | HISTOL./ | UP | |
| ID | AVG AREA | INTENS. | AVG INTENS | TELOM. | CYTOL. | AGE | M/F? | CELLS | PLOIDY | 5C | HISTOL | HISTOR. | DATE | FOLLOW_UP |
| 1 | 18567.30 | 88175.00 | 4.77 | Aug. 01, 2002 | High grade | 53 | M | 25 | Aneupl. | 52.4 | Sep. 04, 2002 | TCC in | Jul. 26, 2004 | No recurrence |
| | | | | | urothelial | | | | | | | situ | | |
| | | | | | CA | | | | | | | | | |
| 2 | 14404.43 | 67863.43 | 4.77 | Jan. 09, 2004 | Degenerative | 59 | M | 25 | Aneupl. | 14.6 | HISTORY | Ductal CA | Aug. 06, 2004 | Orchiectomy |
| | | | | | atypical | | | | | | | of prostate | | |
| | | | | | cells c/w | | | | | | | | | |
| | | | | | with high | | | | | | | | | |
| | | | | | grade CA | | | | | | | | | |
| 3 | 10670.00 | 47318.00 | 4.44 | Apr. 09, 2004 | Atypical | 79 | M | 18 | | | May 24, 2004 | chronic | | NFU |
| | | | | | urothelial | | | | | | | inflammation, | | |
| | | | | | cells c/w | | | | | | | no | | |
| | | | | | high grade | | | | | | | tumor | | |
| | | | | | urothelial | | | | | | | | | |
| | | | | | CA | | | | | | | | | |
| 4 | 8629.71 | 43817.00 | 5.12 | Apr. 16, 2004 | Urothelial | 58 | M | 18 | Aneupl. | 9.9 | May 04, 2004 | Reactive | Aug. 23, 2004 | Cystoscopy |
| | | | | | CA, high | | | | | | | hyperplasia & | | slight irregular |
| | | | | | grade | | | | | | | chronic | | urothelial |
| | | | | | | | | | | | | inflammation, | | mucosa, |
| | | | | | | | | | | | | no | | Cytology high |
| | | | | | | | | | | | | tumor | | grade TCC |
| 5 | 30155.00 | 146899.30 | 4.95 | Apr. 19, 2004 | Atypical | 71 | M | 17 | Aneupl. | 11.1 | May 04, 2004 | chronic | Aug. 03, 2004 | Cystoscopy Pap |
| | | | | | urothelial | | | | | | | inflammation, | | TCC |
| | | | | | cells c/w | | | | | | | no | | |
| | | | | | high grade | | | | | | | tumor | | |
| | | | | | urothelial | | | | | | | | | |
| | | | | | CA | | | | | | | | | |
| 6 | 14086.82 | 66026.45 | 4.66 | Feb. 23, 2004 | TCC, high | 68 | M | 16 | Aneupl. | 10.6 | Oct. 25, 2002 | TCC in | Jun. 21, 2004 | Urothelium with |
| | | | | | grade | | | | | | | situ | | mild dysplasia |
| 7 | 25029.27 | 130259.30 | 5.05 | Apr. 13, 2004 | TCC, high | 69 | F | 25 | Aneupl. | 20.5 | May 18, 2004 | chronic | Aug. 20, 2004 | No tumor, |
| | | | | | grade | | | | | | | inflammation, | | Cytology high- |
| | | | | | | | | | | | | no | | grade TCC |
| | | | | | | | | | | | | tumor | | |
| 8 | 15120.13 | 55427.38 | 3.65 | Dec. 02, 2002 | Atypical | 80 | M | 9 | Broad | 0.9 | Dec. 02, 2002 | Urothelial | Apr. 02, 2004 | No evidence of |
| | | | | | urothelial | | | | Diploid | | | hyperplasia & | | disease |
| | | | | | cells | | | | | | | chronic | | |
| | | | | | suspicious | | | | | | | inflammation | | |
| | | | | | for | | | | | | | | | |
| | | | | | urothelial | | | | | | | | | |
| | | | | | CA | | | | | | | | | |
| 9 | 6865.80 | 30918.60 | 4.50 | Feb. 27, 2003 | Papillary | 68 | M | 0 | Aneupl. | 0.8 | Mar. 07, 2003 | Non | Jun. 13, 2003 | Died of disease |
| | | | | | clusters | | | | | | | papillary | | |
| | | | | | with mild | | | | | | | TCC, | | |
| | | | | | atypia | | | | | | | grade 3 | | |
| | | | | | suspicious | | | | | | | | | |
| | | | | | for | | | | | | | | | |
| | | | | | urothelial | | | | | | | | | |
| | | | | | carcinoma | | | | | | | | | |
| 10 | 12748.33 | 57860.33 | 4.55 | May 16, 2003 | Atypical | 63 | M | 0 | Tetrapl. | 1.8 | HISTORY | Papillary | Aug. 02, 2004 | Pap TCC grade |
| | | | | | papillary | | | | | | | TCC grade | | 2, non-Invasive |
| | | | | | urothelial | | | | | | | 2, non- | | |
| | | | | | cells | | | | | | | invasive | | |
| | | | | | suspicious | | | | | | | | | |
| | | | | | for | | | | | | | | | |
| | | | | | recurrent | | | | | | | | | |
| | | | | | low grade | | | | | | | | | |
| | | | | | CA | | | | | | | | | |
| 11 | 15318.50 | 76309.88 | 4.97 | Jun. 02, 2003 | Degenerative | 63 | M | 0 | Diploid | 0 | May 31, 2003 | TCC grade | Aug. 11, 2004 | No recurrent |
| | | | | | atypical | | | | | | Hx | 3, non- | | disease |
| | | | | | cells | | | | | | | invasive | | |
| | | | | | suspicious | | | | | | | | | |
| | | | | | for CA | | | | | | | | | |
| 12 | 14063.00 | 65695.00 | 4.77 | Jul. 29, 2003 | Highly | 79 | M | 10 | Aneupl. | 32.7 | HISTORY | Papillary | May 25, 2004 | TCC in Situ |
| | | | | | atypical | | | | | | | TCC grade | | |
| | | | | | cells, | | | | | | | 2, invasive | | |
| | | | | | suspicious | | | | | | | | | |
| | | | | | for CA | | | | | | | | | |
| 13 | 5801.33 | 39596.42 | 6.86 | Jun. 04, 2003 | No | 55 | F | 0 | Broad | 5.5 | HISTORY | Breast CA, | | HPV |
| | | | | | malignant | | | | Diploid | | | vaginal | | CONTAMINATION |
| | | | | | cells | | | | | | | contamination | | |
| | | | | | | | | | | | | by | | |
| | | | | | | | | | | | | HPV | | |
| 14 | 8447.14 | 45780.14 | 5.48 | Jul. 25, 2003 | No | 61 | M | 2 | Broad | 1.9 | Jul. 30, 2003 | Papillary | Mar. 12, 2004 | No reurrence |
| | | | | | malignant | | | | Diploid | | | CA of | | |
| | | | | | cells | | | | | | | Renal | | |
| | | | | | | | | | | | | pelvis | | |
| 15 | 12644.40 | 73210.20 | 5.73 | Feb. 06, 2004 | No | 83 | M | 1 | Diploid | 0 | Oct. 29, 2003 | Papillary | Aug. 10, 2004 | No recurrence |
| | | | | | malignant | | | | | | | TCC grade | | |
| | | | | | cells | | | | | | | 3, non- | | |
| | | | | | | | | | | | | invasive | | |
| 16 | 8846.42 | 47386.50 | 5.40 | Jan. 06, 2004 | Papillary | 57 | M | 0 | Hyper- | 0.5 | Jan. 14, 2004 | Spindle | Jun. 01, 2004 | No recurrence |
| | | | | | lesion with | | | | dipl. | | | cell | | |
| | | | | | degenerative | | | | | | | sarcoma | | |
| | | | | | changes | | | | | | | c/w | | |
| | | | | | | | | | | | | synovial | | |
| | | | | | | | | | | | | sacoma in | | |
| | | | | | | | | | | | | kidney & | | |
| | | | | | | | | | | | | adrenal | | |
| | | | | | | | | | | | | glands | | |
| 17 | 7304.29 | 39180.86 | 5.51 | | No | 48 | F | | | | | | | |
| | | | | | malignant | | | | | | | | | |
| | | | | | cells | | | | | | | | | |
| 18 | 6601.71 | 40497.71 | 6.31 | | No | 50 | F | | | | | | | |
| | | | | | malignant | | | | | | | | | |
| | | | | | cells | | | | | | | | | |
| 19 | 6235.67 | 44161.33 | 7.15 | | No | 26 | M | | | | | | | |
| | | | | | malignant | | | | | | | | | |
| | | | | | cells | | | | | | | | | |
| 20 | 6595.27 | 44478.55 | 6.74 | | | | | | | | | | | |
| 21 | 7349.64 | 43855.71 | 6.02 | | | | | | | | | | | |
| 22 | 41565.33 | 227105.67 | 5.26 | Feb. 06, 2004 | Rare | 64 | M | 1 | Diploid | 0 | Feb. 06, 2004 | sessile | Jun. 01, 2004 | Erythema at |
| | | | | | degenerated | | | | | | | tumor | | bladder neck |
| | | | | | atypical | | | | | | | | | but no tumor |
| | | | | | cells in | | | | | | | | | |
| | | | | | background | | | | | | | | | |
| | | | | | of acute | | | | | | | | | |
| | | | | | inflammation | | | | | | | | | |
| 23 | 25665.40 | 143472.40 | 5.51 | Jan. 23, 2004 | Cellular | 80 | M | 2 | Polypl. | 13.1 | Jan. 23, 2004 | No | Jan. 23, 2004 | No recurrent |
| | | | | | changes | | | | | | | recurrent | | disease |
| | | | | | c/w | | | | | | | disease | | |
| | | | | | polyoma | | | | | | | | | |
| | | | | | virus | | | | | | | | | |

As presented in Table 3, generally the shorter the telomere, the greater the chance of the sample comprising a tumor cell or predicting development of cancer. Also, the greater the chance of having invasive (which may also be referred to as high-grade) bladder cancer.

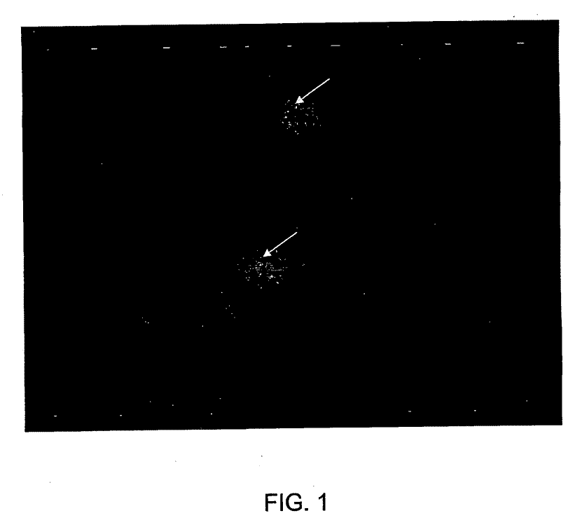

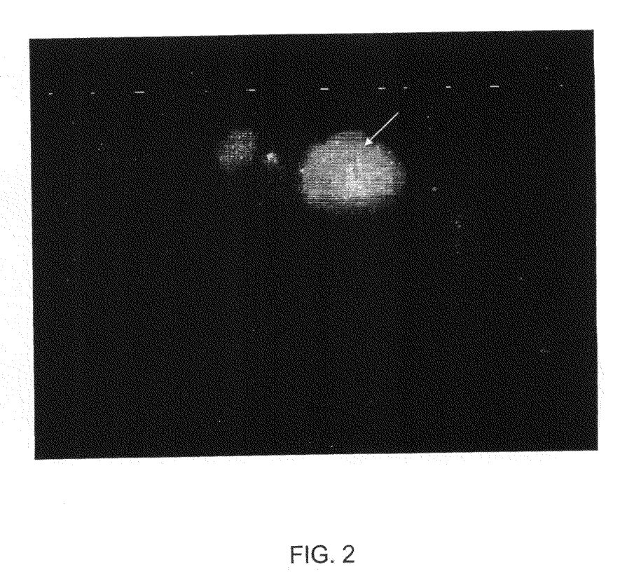

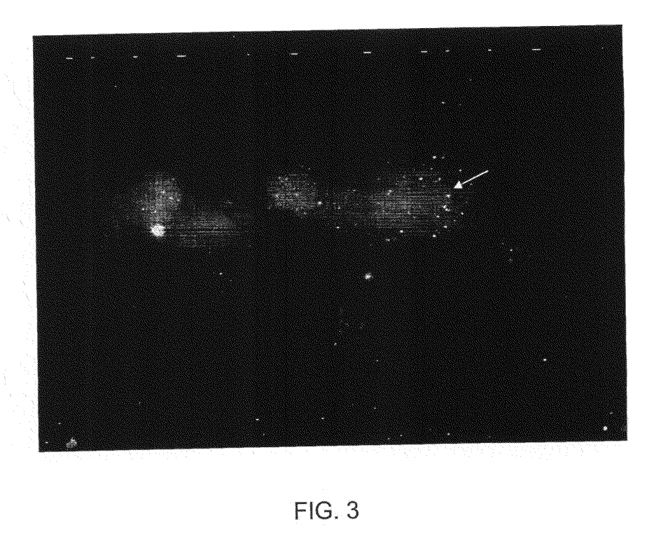

FIG. 1 shows telomere fluorescence of a normal control sample having a telomere length average intensity of 7.15. The normal patient has longer telomeres compared to patients with urothelial carcinoma. FIGS. 2 and 3 show samples having polyoma virus infection wherein the telomere length average intensity was 5.51. By UroVysion FISH analysis, only 2 of 25 cells were abnormal. The sample history was identified as transitional cell carcinoma grade 2.

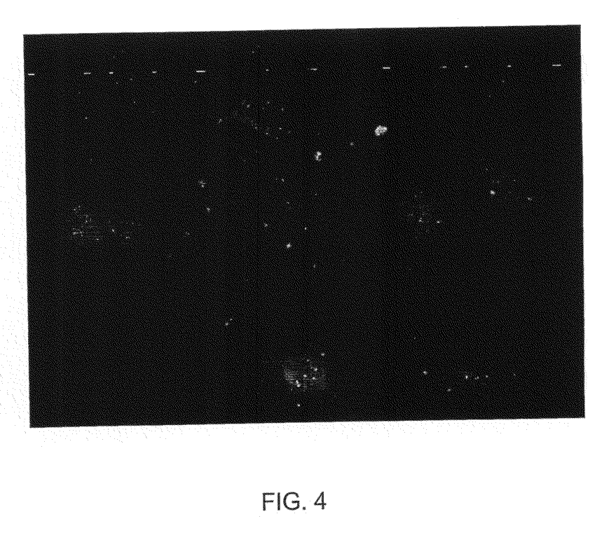

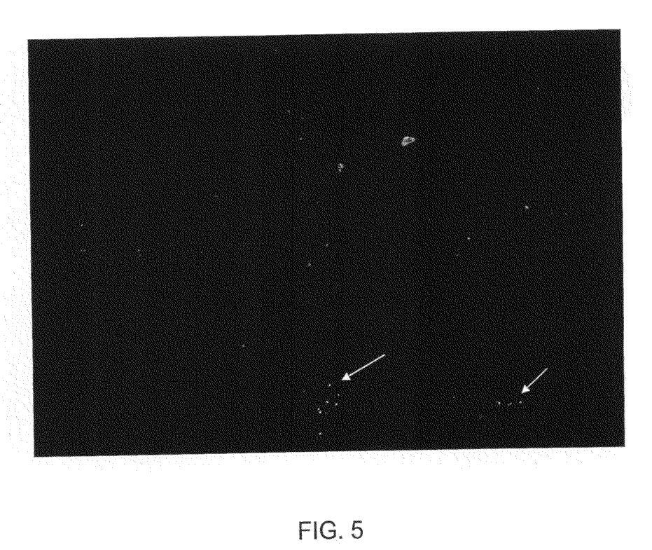

FIG. 4 provides samples of atypical cells consistent with high-grade transitional cell carcinoma (based on only a few abnormal cells), wherein the telomere length average intensity was 4.86 (shortest telomeres compared to controls and Polyoma virus patients). With UroVysion FISH analysis, 18 of 25 cells were identified as abnormal cells. The history of the sample was carcinoma in situ. FIG. 5 demonstrates cytology of transitional cell carcinoma cells, wherein the telomere length average intensity is 4.44. With UroVysion FISH, 18 of 25 cells were classified abnormal. The sample history was transitional cell carcinoma grade 2.

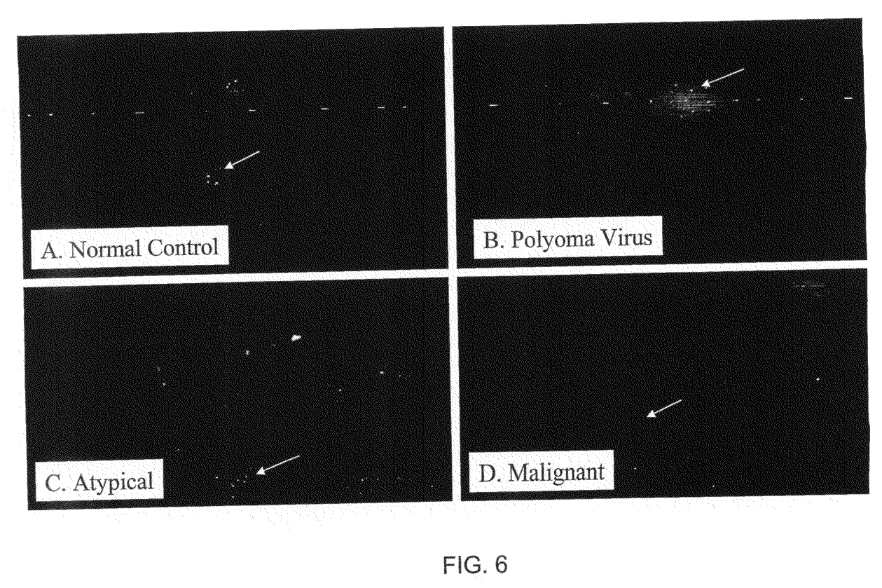

FIGS. 6A-6D show a composite photomicrograph presenting different cytological classifications with corresponding telomere FISH staining. The malignant cells have much dimmer signal compared to the normal cells.

### Example 4

### Telomere Length in Lung Cancer

The present inventors compared telomere length between samples having normal cells and samples having cancerous cells. Specifically, samples comprising bronchial brush specimens were obtained upon bronchoscopic retrieval of lung tissue using a bronchial brush and detachment of the attached material. More specifically, telomere length was compared in normal bronchial brushes, tumor bronchial brushes, and tumor touch preparations. Telomere length was also correlated with the expression of the 5p gene (hTERT).

Twenty samples of patients with lung cancer were analyzed for telomere length in normal bronchial brushes (NBB), which represent tissue from the side of the lung other than the side with the tumor; tumor bronchial brushes (TBB), which represent tissue from the side of the lung with the tumor; and tumor touch preparations (TIP). The data is provided in Table 4.

TABLE 4
Telomeres and Lung Cancer

| | Smoking | | | | | | | | |
|---|---|---|---|---|---|---|---|---|---|
| Lab | History | | | | | | | | |
| Number | (pack/year) | | Age | NBB | TBB | TTP | TTP 5p | Histological Diagnosis | Follow up |
| SP02- | 1 | pack/day | 57 | 4.44 | 4.37 | 4.54 | 1.13 | Poorly differentiated non-small | Metastasis to brain |
| 015 | | | | | | | | cell carcinoma | (July 2004) |
| SP02- | 75 | | 62 | 3.87 | 4.31 | 4.48 | 1.67 | Basiloid Squamous Carcinoma. | Relapsed |
| 018 | | | | | | | | Vascular/lymphatic invasion | (Feb. 18, 2003) |
| SP02- | 80 | | 69 | 4.024 | 5.02 | 4.48 | 1.23 | Poorly differentiated | No recurrence |
| 020 | | | | | | | | adenocarcinoma. Vascular | |
| | | | | | | | | invasion | |
| SP02- | 0.5 | | 73 | 3.69 | 4.06 | 4.61 | 1.56 | Moderately differentiated | No recurrence |
| 023 | | | | | | | | adenocarcinoma | |
| SP02- | 50 | | 71 | 3.88 | 4.65 | 4.33 | 1.01 | Well differentiated squamous | No recurrence |
| 027 | | | | | | | | cell carcinoma | |
| SP02- | 9.5 | | 74 | 4.15 | 4.39 | 5.70 | 1.00 | Poorly differentiated squamous | Metastasis |
| 028 | | | | | | | | cell carcinoma | (Apr. 21, 2004) |
| SP03- | 5 | | 60 | 5.29 | 5.84 | 7.34 | 1.62 | Non-small cell lung carcinoma/ | Remision |
| 001 | | | | | | | | favor squamous cell carcinoma. | |
| SP03- | 0 | | 55 | 4.98 | 5.78 | 5.78 | 1.35 | Poorly differentiated | No recurrence |
| 003 | | | | | | | | adenocarcinoma. | |
| SP03- | 0 | | 84 | 4.51 | 5.05 | 4.64 | . | Adenocarcinoma of the right | No recurrence |
| 005 | | | | | | | | lower lobe with bronchioalveolar | |
| | | | | | | | | features. | |
| SP03- | 0 | | 68 | 5.70 | 5.36 | 5.80 | 2.90 | Well differentiated | No recurrence |
| 006 | | | | | | | | adenocarcinoma. | |
| SP03- | 66 | | 65 | 5.23 | 5.38 | 5.54 | 1.40 | Very poorly differentiated | Spread to splean |
| 007 | | | | | | | | features suggestive of | (Jul. 16, 2004) |
| | | | | | | | | squamous cell carcinoma | |
| SP03- | 130 | | 76 | 5.26 | 4.64 | 3.95 | 0.98 | Moderately differentiated | No recurrence |
| 009 | | | | | | | | squamous cell carcinoma. | |
| SP03- | 0 | | 81 | 4.90 | 5.61 | 5.01 | 1.22 | Adenocarcinoma of the left | No recurrence |
| 010 | | | | | | | | lower lung. | |
| SP03- | 30 | | 73 | 5.35 | 4.94 | 8.08 | 0.94 | Moderately to poorly | No recurrence |
| 011 | | | | | | | | differentiated adenocarcinoma. | |
| SP03- | 36 | | 54 | 4.46 | 4.47 | 5.62 | 1.05 | Moderately to poorly | Relapsed in |
| 012 | | | | | | | | differentiated adenocarcinoma. | Jun. 22, 2004 |
| SP03- | 50 | | 70 | 3.28 | 4.76 | 5.44 | 0.96 | Moderately differentiated | No recurrence |
| 013 | | | | | | | | adenocarcinoma | |
| SP03- | 61.5 | | 63 | 4.43 | 4.40 | 6.13 | 1.00 | Poorly differentiated squamous | Relapsed |
| 014 | | | | | | | | cell carcinoma | (Nov. 25, 2003) |
| SP03- | 135 | | 70 | 3.92 | 4.21 | 3.83 | 1.30 | Poorly differentiated | Metastasis |
| 015 | | | | | | | | adenocarcinoma. | (Jun. 08, 2004) |
| SP04- | 75 | | 74 | 3.43 | 3.49 | 4.93 | 1.43 | Moderately differentiated | Relapsed in Apr. 07, 2004 |
| 001 | | | | | | | | adenocarcinoma | |
| SP04- | ½ | pack/day | 69 | 3.92 | 4.83 | 4.88 | 1.05 | Poorly differentiated squamous | No recurrence |
| 002 | | | | | | | | cell carcinoma | |

The column “TTP 5p” represents a probe specific for hTERT performed on the tumor touch preparation, which gene is located on chromosome 5p, and this refers to the ratio of the gene for hTERT in relationship to the centromeric region of chromosome 5. If the value of the ratio is >1, then 5p is amplified.”

As presented therein, 14 of 20 TTP are the longest telomeres, and 13 TBB of 20 are longer than NBB.

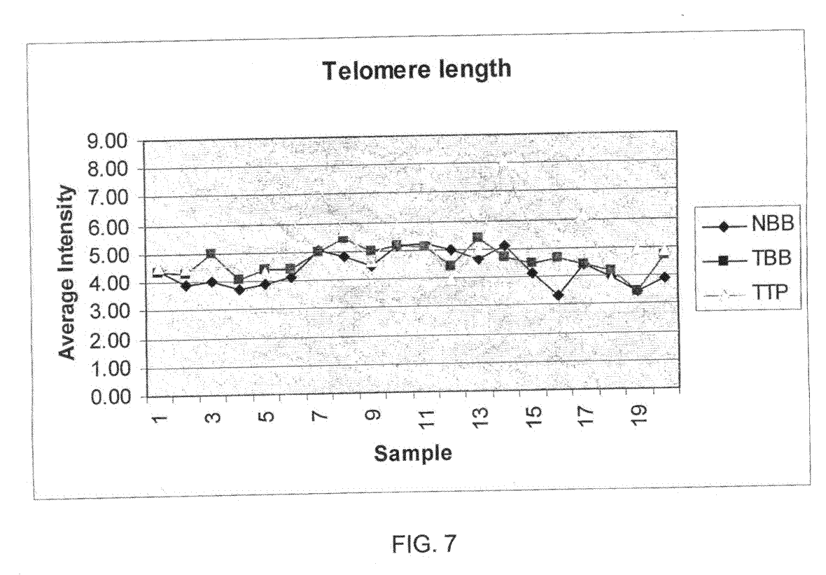

The samples were analyzed by determining the average intensity corresponding to telomere length. The average intensity was determined by measuring the area of the nuclear fluorescence over the signal of the telomere length. The area of the nucleus may be determined by nuclear stain, such as DAPI. The obtained results are presented in FIG. 7. Telomere length in tumor touch preparation (yellow line) in most cases is higher then in TBB and NBB. TL in TBB is generally intermediate between TTP and NBB; if ratio of TTP:NBB in bronchial brush is >1, could be used to predict that mass in lung is malignant. This may be due to a field effect in which telomeres lengthen in the bronchial epithelial cells on the side of the tumor. The average telomere length, as interpreted by the measured median average intensity, for NBB is 4.33; for TBB is 4.63, and for TTP is 5.25.

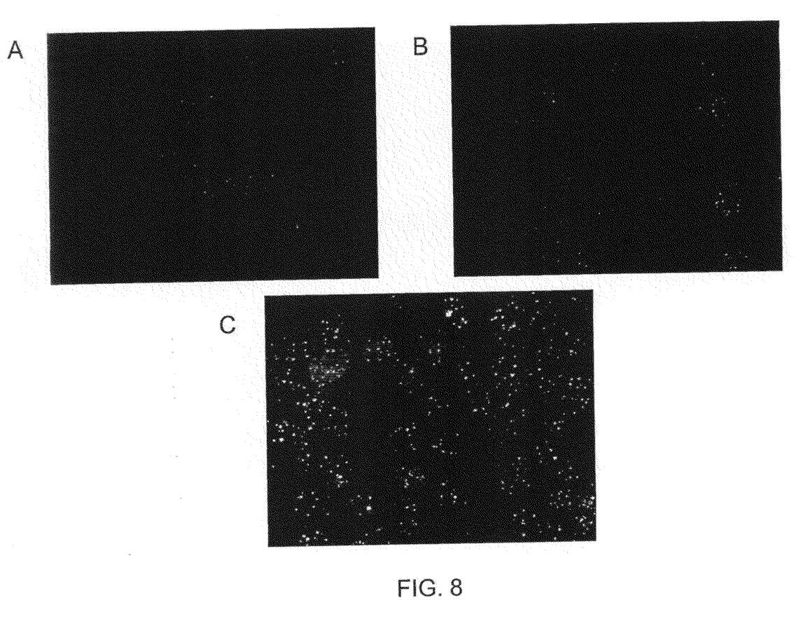

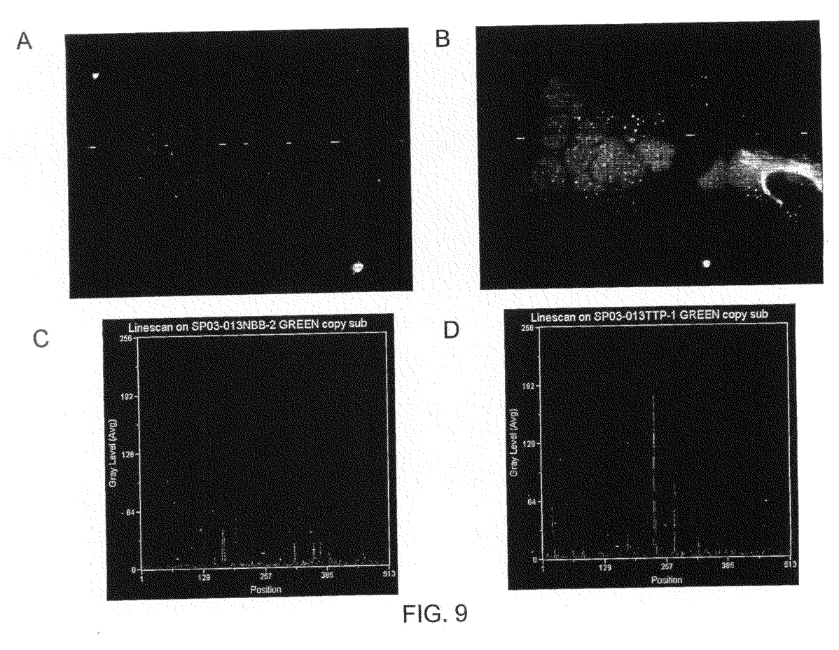

FIGS. 8A-8C demonstrate representative examples of NBB (FIG. 8A), TBB (FIG. 8B), and TTP (FIG. 8C) fluorescence. FIGS. 9A-9D show additional representative examples of NBB (FIG. 9A) and TTP (FIG. 9B) and includes their respective linescans (FIGS. 9C and 9D), which represents the telomeric signals in the image. Note in FIG. 9D that the fluorescent signal intensity on the x-axis is increased compared to FIG. 9C.

Table 5 provides a comparison of smoking history and telomere length of tumor touch preps. The greater the number of packs/yr smoked, the shorter the telomeres. Also, the older the patient, the shorter the telomeres.

TABLE 5
Telomere Length in TTP for Smokers

| Smoking History (pack/year) | TTP telomere length |
|---|---|
| 75 | 4.93 |
| 80 | 4.48 |
| 130 | 3.95 |
| 135 | 3.83 |

The TTP telomeres divided into long and short telomeres. For 9 cases of long TTP telomeres (TL>5.25), 6 relapsed and 3 had no recurrence. For 11 of the short TTP telomeres, only 2 relapsed whereas 9 had no recurrence. Thus, the longer the telomeres in the tumor, the worse the prognosis. Therefore, the quantitative FISH methods of the present invention are useful for prognosticating the development and/or severity of cancer.

Furthermore, in general telomeres from cells on the non-affected side of the lung are shorter than those on the affected side, consistent with there being a field effect surrounding the tumor cells having long telomeres.

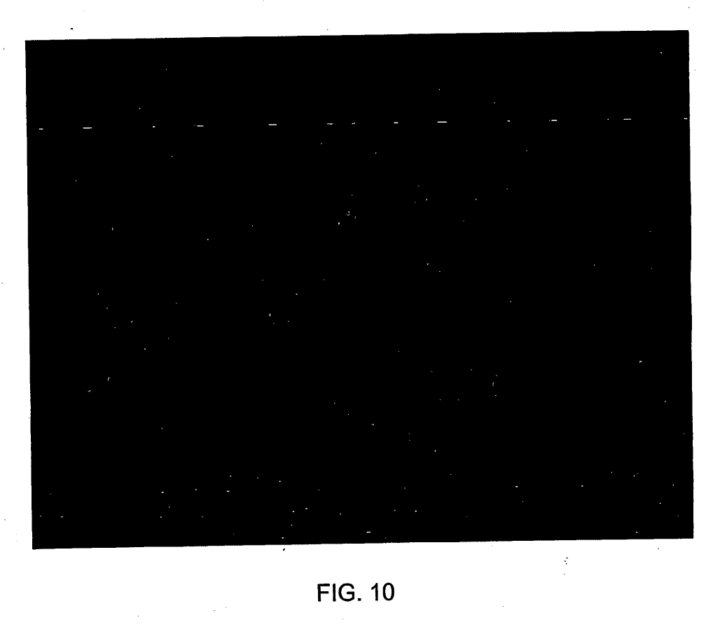

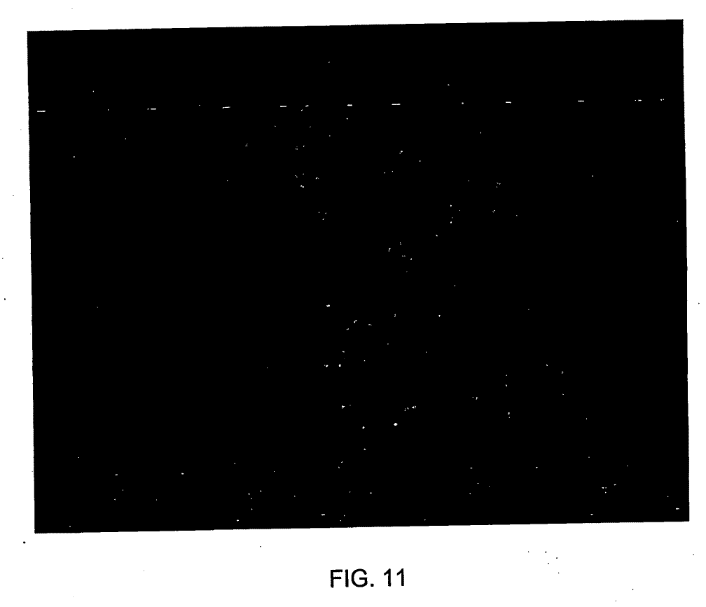

FIGS. 10 and 11 show images identifying the presence of chromosome 5p, which represents the hTERT locus. The hTERT is greatly amplified here based upon many more green than red signals (shows in a color photo).

### Example 5

### Telomere Length in Lymphoma

In particular aspects of the invention, the methods and compositions herein are useful for any kind of lymphoma, including Non-Hodgkin's B-cell Lymphomas. It is considered that in diploid low-grade lymphomas the telomeres become shorter with each cell division, eventually leading to chromosomal instability and fusion, ultimately lead to transformation to a higher grade lymphoma. Upon transformation of the lymphocyte, the aneuploid (or even diploid) cells regain their telomere length through re-activation of telomerase. They have increased proliferation and may be refractory to therapy.

The telomeric length in non-Hodgkin's lymphoma was determined by inventive methods. Fine needle aspirates were assayed with FITC-labeled peptide nucleic acid (PNA) telomeric FISH probe and counterstained with propidium iodide. Digital fluorescence microscopy as used to capture and quantitate the average telomeric fluorescent intensity per pixel as an indirect measurement of telomere length. The samples from the individuals being assayed were follicular lymphoma (Grade I, II, or III); small lymphocytic lymphoma, small lymphocytic lymphoma (transformed), large cell lymphoma, mantle cell lymphoma, and marginal zone lymphoma. The lymph node samples included those from neck lymph node/soft tissue; head/chest/lung soft tissue; axilla/supraclavicular lympho node; abdominal lymph node/soft tissue; retroperitoneal lymph node; inguinal lymph node/kidney; and pelvis soft tissue.

A Leica automated image system with OPENLAB software was employed. A macro was built to acquire a stack of images under the same exposure condition. Gain and offset were carefully determined to avoid signal saturation. Merged images had been stored as 8 bit RGB pseudo-colored images. For telomere signal processing, following the counterstain a region of interest is defined, after which linescans are performed and through which backgrounds can be subtracted. Table 6 below provides quantitative FISH results for the samples. Region Label represents the location and the number of cells analyzed in that image. This particular image comprises a tumor sample.

| | | Integrated | Average | Minimum | Maximum |
|---|---|---|---|---|---|
| Region Label | Area | Intensity | Intensity | Intensity | Intensity |
| 32 | 6518 | 103787 | 15.92 | 0 | 191 |
| 20 | 7323 | 116425 | 15.90 | 0 | 190 |
| 29 | 8362 | 161456 | 19.31 | 0 | 189 |
| 71 | 8525 | 159093 | 18.66 | 0 | 177 |
| 21 | 8770 | 133883 | 15.27 | 1 | 192 |
| 7 | 8962 | 170141 | 18.98 | 0 | 187 |
| 60 | 9246 | 181275 | 19.61 | 0 | 190 |
| 22 | 9397 | 177486 | 18.89 | 0 | 194 |
| 26 | 9456 | 171185 | 18.10 | 0 | 177 |
| 8 | 9769 | 158782 | 16.25 | 0 | 194 |
| 34 | 10257 | 180823 | 17.63 | 0 | 185 |
| 33 | 10513 | 189586 | 18.03 | 0 | 183 |
| 49 | 11426 | 207027 | 18.12 | 0 | 193 |
| 23 | 12093 | 226264 | 18.71 | 0 | 174 |
| 44 | 12131 | 224338 | 18.49 | 0 | 185 |
| 46 | 13343 | 225737 | 16.92 | 0 | 196 |
| 36 | 14876 | 279174 | 18.77 | 0 | 188 |
| | | | 17.86 | | |

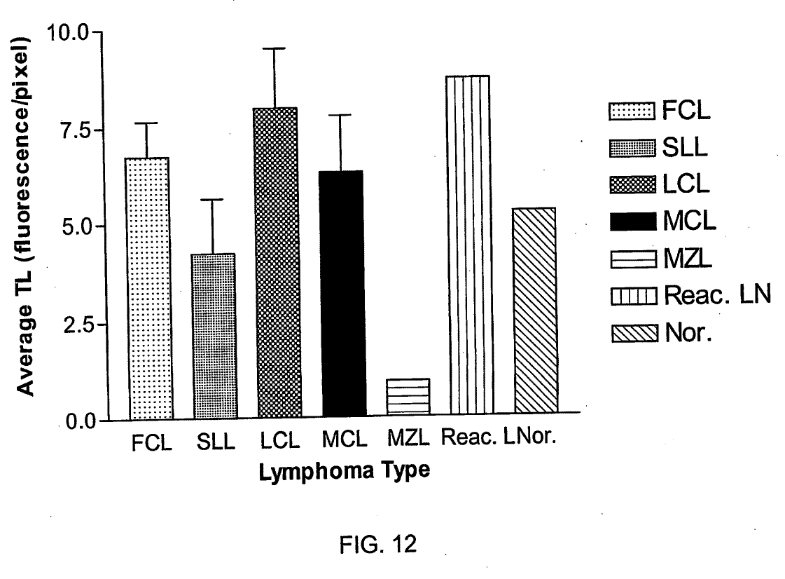

FIG. 12 shows telomere length in different subtypes of lymphoma. Low-grade lymphomas (not yet transformed) generally are characterized by shorter TL.

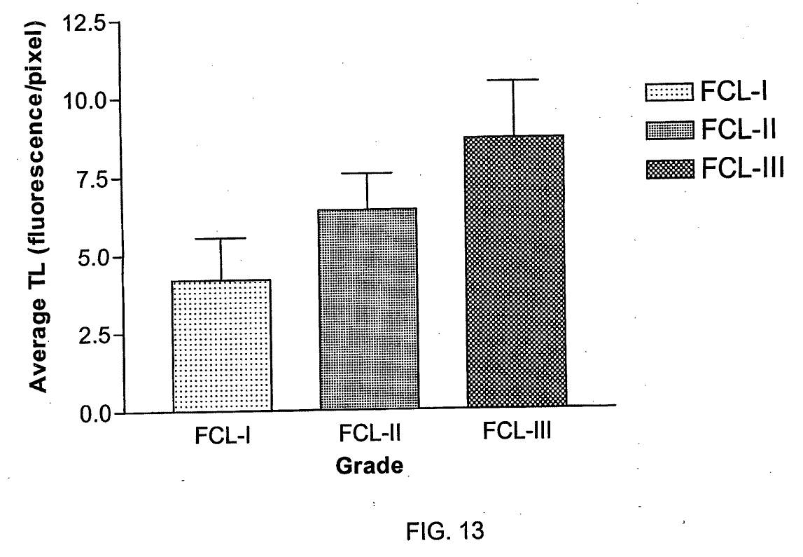

FIG. 13 shows telomere length for different grades of FCL—Grade I, Grade II, or Grade III. With increasing grade and severity of the lymphoma, telomeres are lengthening.

As shown below, telomere length correlated with DNA ploidy and proliferation with aneuploid lymphomas and lymphomas with high Ki-67 having significantly longer telomere length. Ki-67 is a nuclear marker for cell proliferation.

| | Number | TL (Mean/SD) | P-value |
|---|---|---|---|
| Ploidy | | | |
| Aneuploid | 6 | 8.31/3.52 | 0.05 |
| Diploid | 20 | 5.36/3.00 | |
| Ki-67 | | | |
| >25% | 12 | 7.23/3.11 | 0.09 |
| ≦25% | 14 | 5.02/3.22 | |

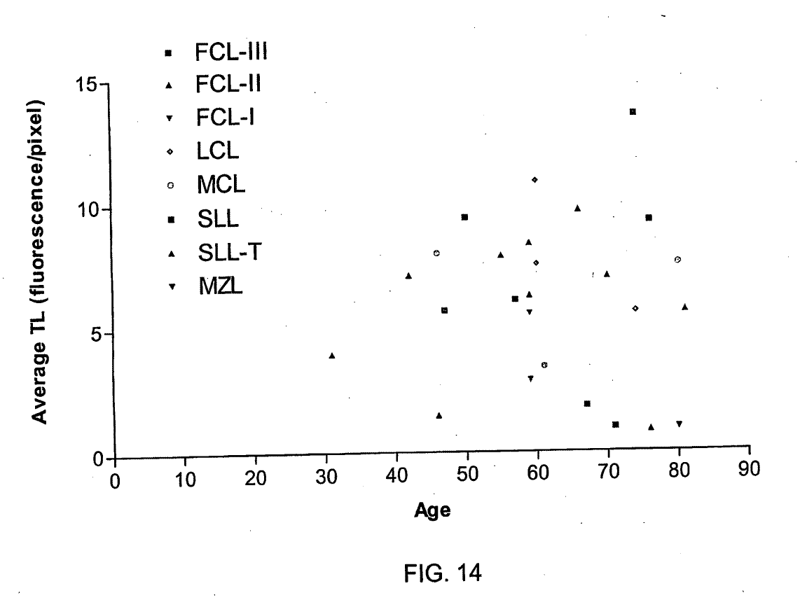

Furthermore, as shown in FIG. 14, telomere length is shown in comparison with age and lymphoma subtypes. Although there was no correlation between patient age and telomere length, histologic subtype does provide a correlation (data not shown). Also, patients that were within 24 months from their initial diagnosis had much longer telomeres than patients with a prolonged clinical course.

| Duration | Number | TL (Mean/SD) | p-value |
|---|---|---|---|
| ≦24 months | 13 | 8.07/2.54 | <0.01 |
| >24 months | 13 | 4.01/2.70 | |

In specific embodiments, there is no correlation between telomere length and relapse following therapy, although in alternative embodiments, there is a correlation. That is, absence of a correlation may be due to the dearth of effective therapy for low-grade lymphomas. In specific embodiments, patients with high-grade lymphomas who relapse after therapy relapse with longer telomeres.

Thus, as shown in this Example, the inventive methods provide a beneficial procedure for routine clinical use for measuring telomere length in lymphoid cells with longer telomeres. In a specific embodiment, telomere length is used as a guide to anti-telomerase therapy, contemplating that patients with longer telomere length may require conventional chemotherapy (and/or other anti-cancer therapies) in addition to anti-telomerase therapy.

### Example 6

### Telomere Length in Other Cancers

The methods described herein are suitable for any cancer. That is, based on the teachings provided herein, one of skill in the art can analogously determine the ratio of the nuclear area to the average telomere length of cells for a particular cancer vs. control cells and identify the trend characteristic for that particular cancer. That is, in some cancers the telomeres will on average be shorter compared to control telomeres, such as with bladder cancer, and in other cancers the telomeres will be on average longer compared to control telomeres, such as with lung cancer.

In specific embodiments of the invention, cancers for which the compositions and methods may be diagnostic, prognostic, or both also include breast cancer, brain cancer, prostate cancer, FNA of thyroid cancer, lung cancer FNA, colon cancer, pancreatic cancer, spleen cancer, stomach cancer, esophageal cancer, ovarian cancer, uterine cancer, testicular cancer, liver cancer, gall bladder cancer, leukemia, melanoma, head and neck cancer, throat cancer, and kidney cancer, for example. In addition, this technique could be used to diagnose malignancy in pleural fluids or ascites fluid, and peritoneal washes such as reactive mesothelial cells versus mesothelioma, reactive mesothelial cells versus adenocarcinoma including ovarian carcinoma, reactive lymphocytes versus lymphoma or any other neoplastic disorder affecting all body cavities that present with effusions including diagnosing malignancies in cerebrospinal fluids, for example.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PATENTS AND PATENT APPLICATIONS

U.S. Pat. No. 5,693,474

U.S. Pat. No. 5,707,795

U.S. Pat. No. 6,054,314

U.S. Pat. No. 6,174,681

U.S. Pat. No. 6,376,188

U.S. Patent Application Publication No. 2002/0160409

WO 97/35871

### PUBLICATIONS

Allsopp et al., 1992, Proc. Natl. Acad. Sci. USA 89:10114-10118.
Blackburn and Szostak, 1984, Ann. Rev. Biochem. 53:163-194.
Blackburn, 1991 Nature 350:569-573.
Catto et al., 2004, BJU Int., 93(1):19-24.
Counter et al., 1992, EMBO 11:1921-1929.
Counter et al., 1994, Proc. Natl. Acad. Sci. USA 91:2900-2940.
Dalquen, P. et al., 2002, Cancer Cytopath. 976:374-9.
Golubovskaya et al., 1999, Mol. Carcinog. 24:209-217.
Halling, K. C. et al., 2002, J. Urol. 167:2001-2006.
Harley et al., 1990, Nature 345:458-460.
Harley, 1991, Mut. Res. 256:271-282.
Hastie et al., 1990, Nature 346:866-868.
Kageyama et al., 1997, Int. J. Urol. 4:407-410.
Katz et al., 1997, Cancer Cytopath. 81:354-364.
Lindsey et al., 1991, Mut. Res. 256:45-8.
Little, 2003, Int. Urol. Nephrol. 35:331-343.
Moyzis et al., 1988, Proc. Natl. Acad. Sci. 85:6622-6626.
Plentz et al., 2004, Hepatol. 40:80-86.
Sarodsy, et al., 2002, J. Urol. 168:1950-1954.
Soloway et al., 1994, Cancer April 1; 73(7):1926-31.
Van Heek et al., 2002, Am. J. Pathol. 161(5):1541-1547.
Varella-Garcia et al., 2004, Urol. Oncol. 22(1):16-19.
Vaziri et al., 1993, Am. J. Human Genetics 52:661-667.
Watson, 1972, Nature New Biology 239:197-201.
Wright and Shay, 1992, Trends Genetics 8:193-197.
Zakian, 1989, Ann. Rev. Genetics 23:579-604.

What is claimed is:

1. A method of diagnosing and/or prognosticating cancer in an individual, comprising the steps of:
providing a sample from the individual, wherein said sample comprises at least one cell;
assaying one or more cells of said sample in situ to determine a telomere length quantity, said quantity comprising a numerical correlation of the telomere length and the area of the nucleus; and
determining said diagnosis or prognosis of the individual based on said quantity.

2. The method of claim 1, wherein the numerical correlation is further defined as the ratio of area of the nucleus to the mean telomere length.

3. The method of claim 1, wherein the area of the nucleus is determined by a nuclear stain.

4. The method of claim 1, wherein the sample is urine, blood, cerebrospinal fluid, pleural fluid, ascites fluid, bladder washings, bronchial brush samples, oral washings, touch preps, cheek scrapings, feces, biopsy, fine needle aspirate, nipple aspirates, urine, sputum, bronchiolar alveolar lavage, pap smears, anal scrapings, skin scrapings, or tissue section.

5. The method of claim 4, wherein the sample is urine.

6. The method of claim 1, wherein said assaying step comprises fluorescence in situ hybridization (FISH).

7. The method of claim 2, wherein when the sample comprises at least one bladder cell and the ratio is less than about 5.1, the sample comprises at least one bladder cancer cell.

8. The method of claim 2, wherein when the sample comprises at least one lung cell and the ratio is greater than about 5.1, the sample comprises at least one lung cancer cell.

9. The method of claim 1, wherein the diagnosis of the cancer is an initial diagnosis for the individual.

10. The method of claim 1, wherein the individual was previously diagnosed with cancer.

11. The method of claim 1, wherein the individual was previously diagnosed as not having cancer.

12. The method of claim 11, further defined as providing the individual with a follow-up diagnosis to the previous non-cancerous diagnosis.

13. The method of claim 1, said method further defined as utilizing a high throughput analysis for said diagnosis and/or prognosis.

14. The method of claim 1, wherein the assaying step is further defined as comprising hybridization of a polynucleotide that targets telomeric DNA.

15. The method of claim 14, wherein the polynucleotide comprises a fluorescent label or a chromagenic label.

16. The method of claim 1, wherein the individual is suspected of having low-grade urothelial or bladder cancer.

17. The method of claim 16, wherein the numerical correlation is further defined as the ratio of area of the nucleus to the mean telomere length.

18. The method of claim 1, wherein the individual is one desired to be tested for a predisposition to developing cancer.

19. The method of claim 18, wherein the cancer is bladder cancer.

20. A kit for determining a diagnosis and/or a prognosis of cancer for an individual, housed in a suitable container and comprising one or more of the following:
one or more telomere-targeting molecules;
a label; and
a nuclear stain.

21. The kit of claim 20, wherein the one or more telomere-targeting molecules comprises a polynucleotide that targets telomeric DNA.

22. The kit of claim 20, wherein the label comprises a fluorophore or a chromagen.

23. The kit of claim 22, wherein the label comprises a fluorophore.

24. The kit of claim 20, further comprising instructions for said kit, wherein the instructions comprise an expected ratio of nuclear area to quantity of telomere length, said ratio indicative of said cancer.

25. The kit of claim 20, further comprising a sample collector.

26. The kit of claim 22, wherein the fluorophore is 7-amino-4-methylcoumarin-3-acetic acid (AMCA), 5- (and -6)-carboxy-X-rhodamine, lissamine rhodamine B, 5-carboxyfluorescein, 6-carboxyfluorescein, fluorescein-5-isothiocyanate (FITC), 7-diethylaminocoumarin-3-carboxylic acid, tetramethylrhodamine-5-isothiocyanate, tetramethylrhodamine-6 isothiocyanate, 5-carboxytetramethylrhodamine, 6-carboxytetramethylrhodamine, 7-hydroxycoumarin-3-carboxylic acid, 6-[fluorescein 5-carboxamido]hexanoic acid, 6-[fluorescein 6-carboxamido]hexanoic acid, N-(4,4-difluoro-5,7-dimethyl-4-bora-3a,4a diaza-3-indacenepropionic acid, eosin-5-isothiocyanate, or erythrosin-5-isothiocyanate.

27. The kit of claim 22, wherein the nuclear stain is DAPI, Hoechst 33342 dye, 7-actinomycin-D/7-Aminoactinomycin D/Chromomycin A3, propidium iodide, or Nuclear fast red.

28. The kit of claim 25, wherein the sample collector is a cup, a toothpick, a loop, a syringe, a bronchial brush, a needle, a cotton swab, or a cyto brush.

29. A method of differentiating a cell having a polyoma virus infection from another cell that does not have a polyoma virus infection, comprising the steps of:
providing at least one cell suspected of having a polyoma virus infection;
assaying one or more cells of said sample in situ to determine a telomere length quantity, said quantity comprising a numerical correlation of the mean telomere length and the area of the nucleus; and
determining whether or not the cell has a polyoma virus infection based on said quantity.

30. The method of claim 29, wherein the numerical correlation is further defined as the ratio of area of the nucleus to the mean telomere length.
